(19) United States
(12) Patent Application Publication
(10) Pub. No.: US 2023/0245741 A1
(43) Pub. Date: Aug. 3, 2023

(54) INFORMATION PROCESSING DEVICE, INFORMATION PROCESSING SYSTEM, AND INFORMATION PROCESSING METHOD

(71) Applicant: SONY GROUP CORPORATION, TOKYO (JP)

(72) Inventors: MAKI SHIGYO, TOKYO (JP); MASANORI KATSU, TOKYO (JP)

(21) Appl. No.: 17/754,489

(22) PCT Filed: Aug. 13, 2020
(86) PCT No.: PCT/JP2020/030758

§ 371 Date: Apr. 4, 2022

(30) Foreign Application Priority Data
Oct. 11, 2019 (JP) 2019-187305

(51) Int. Cl. G16H 20/10; G16H 50/20

(52) U.S. Cl. CPC G16H 20/10; G16H 50/20

(57) ABSTRACT

Provided is an information processing device including a physical condition index acquisition unit (132) that acquires, from a monitor device that monitors one or more physical condition indexes on a user, the physical condition indexes, an attribute information acquisition unit (134) that acquires attribute information on the user, a medication information acquisition unit (138) that acquires a medication information on the user, a comparison unit (140) that compares the physical condition indexes with a preset first threshold value, an evaluation unit (144) that refers to a history of the physical condition indexes on the user or another user selected based on at least one of the attribute information and the medication information according to a result of the comparison to evaluate the physical condition indexes, and an output unit (190) that outputs predetermined information according to a result of the evaluation.

### FIELD

The present disclosure relates to an information processing device, an information processing system, and an information processing method.

### BACKGROUND

As the future shortage of doctors is predicted, various treatment support systems have been developed to support treatment. For example, as one of the above-mentioned treatment support systems, there may be a treatment support system that determines whether a patient undergoing home treatment should visit a medical institution.

### CITATION LIST

### Patent Literature

Patent Literature 1: JP 2000-116607 A

### SUMMARY

### Technical Problem

However, while recognizing the effectiveness of the current treatment support system (information processing device), the present inventors have studied to further enhance the effectiveness of the treatment support system. Therefore, the present disclosure proposes an information processing device, an information processing system, and an information processing method that are more effective.

### Solution to Problem

According to the present disclosure, an information processing device is provided. The information processing device includes: a physical condition index acquisition unit that acquires, from a monitor device that monitors one or more physical condition indexes of a user, the physical condition indexes; an attribute information acquisition unit that acquires attribute information on the user; a medication information acquisition unit that acquires a medication information on the user; a comparison unit that compares the physical condition indexes with a preset first threshold value; an evaluation unit that refers to a history of the physical condition indexes of the user or another user selected based on at least one of the attribute information and the medication information according to a result of the comparison to evaluate the physical condition indexes; and an output unit that outputs predetermined information according to a result of the evaluation.

Also, according to the present disclosure, an information processing system is provided. The information processing system includes: a monitor device that monitors one or more physical condition indexes on a user; and an information processing device. In the information processing system, the information processing device includes: a physical condition index acquisition unit that acquires, from the monitor device, the physical condition indexes, an attribute information acquisition unit that acquires attribute information on the user, a medication information acquisition unit that acquires medication information on the user, a comparison unit that compares the physical condition indexes with a preset first threshold value, an evaluation unit that refers to a history of the physical condition indexes on the user or another user selected based on at least one of the attribute information and the medication information according to a result of the comparison to evaluate the physical condition indexes, and an output unit that outputs predetermined information according to a result of the evaluation.

Moreover, according to the present disclosure, an information processing method is provided. The information processing method includes: acquiring, from a monitor device that monitors one or more physical condition indexes on a user, the physical condition indexes; acquiring attribute information on the user; acquiring a medication information on the user; comparing the physical condition indexes with a preset first threshold value; referring to a history of the physical condition indexes on the user or another user selected based on at least one of the attribute information and the medication information according to a result of the comparison to evaluate the physical condition indexes; and outputting predetermined information according to a result of the evaluation.

### BRIEF DESCRIPTION OF DRAWINGS

FIG. 1 is a flowchart illustrating a user's usage procedure in a treatment support system.

FIG. 2 is a system diagram illustrating a schematic functional configuration of a treatment support system 1 according to a first embodiment of the present disclosure.

FIG. 3 is a diagram illustrating a functional configuration of a server 10 according to the embodiment.

FIG. 4 is a diagram illustrating a functional configuration of a monitor determination block 120 according to the embodiment.

FIG. 5 is a diagram illustrating a functional configuration of an evaluation block 130 according to the embodiment.

FIG. 6 is a diagram illustrating a functional configuration of a monitor device 30 according to the embodiment.

FIG. 7 is a diagram illustrating an external example of a monitor device 30a according to the embodiment.

FIG. 8 is a flowchart illustrating an information processing method according to the embodiment.

FIG. 9 is an explanatory diagram illustrating an example of a login screen 800 according to the embodiment.

FIG. 10 is an explanatory diagram illustrating an example of an input screen 806 according to the embodiment.

FIG. 11 is an explanatory diagram illustrating an example of a management screen 810 according to the embodiment.

FIG. 12 is an explanatory diagram illustrating an example of a monitor item setting screen 812 according to the embodiment.

FIG. 13 is an explanatory diagram illustrating an example of a monitor device management screen 816 according to the embodiment.

FIG. 14 is an explanatory diagram (No. 1) illustrating an example of a determination screen 818 according to the embodiment.

FIG. 15 is an explanatory diagram (No. 1) for explaining an evaluation method according to the embodiment.

FIG. 16 is an explanatory diagram (No. 2) for explaining an evaluation method according to the embodiment.

FIG. 17 is an explanatory diagram (No. 3) for explaining an evaluation method according to the embodiment.

FIG. 18 is an explanatory diagram illustrating an example of an output screen 820 according to a modification of the embodiment.

FIG. 19 is an explanatory diagram illustrating an example of an output screen 824 according to a modification of the embodiment.

FIG. 20 is an explanatory diagram illustrating an example of a setting screen 826 according to a modification of the embodiment.

FIG. 21 is an explanatory diagram (No. 2) illustrating an example of the determination screen 818 according to a modification of the embodiment.

FIG. 22 is a diagram illustrating a functional configuration of an estimation block 160 according to a second embodiment of the present disclosure.

FIG. 23 is an explanatory diagram (No. 1) for explaining an estimation method according to the embodiment.

FIG. 24 is an explanatory diagram for explaining a method of estimating an ingested nutritional component according to the embodiment.

FIG. 25 is an explanatory diagram illustrating an example of an activity level display screen 838 according to the embodiment.

FIG. 26 is an explanatory diagram (No. 2) for explaining the estimation method according to the embodiment.

FIG. 27 is a flowchart of a first example of the embodiment of the present disclosure.

FIG. 28 is a flowchart of a second example of the embodiment of the present disclosure.

FIG. 29 is a hardware configuration diagram illustrating an example of a computer that implements functions of an image processing device.

### DESCRIPTION OF EMBODIMENTS

Hereinafter, preferred embodiments of the present disclosure will be described in detail with reference to the accompanying drawings. Note that, in the present specification and drawings, redundant description of a component having substantially the same functional configuration is omitted by providing the same reference sign. In addition, in this specification and drawings, similar components of different embodiments may be distinguished by adding different alphabets after the same reference signs. However, when there is no particular need to distinguish between similar components, only the same reference signs are given.

The description will be given in the following order.

1. Background leading to the creation of the present embodiment

2. First Embodiment

2.1 Schematic configuration of treatment support system 1

2.2 Detailed configuration of server 10

2.3 Detailed configuration of monitor determination block 120

2.4 Detailed configuration of evaluation block 130

2.5 Detailed configuration of monitor device 30

2.6 Information processing method

2.7 Modifications

3. Second Embodiment

3.1 Detailed configuration of estimation block 160

3.2 Information processing method

4. Example

4.1 First example

4.2 Second example

5. Summary

6. Hardware configuration

7. Supplement

### 1. BACKGROUND LEADING TO THE CREATION OF THE PRESENT EMBODIMENT

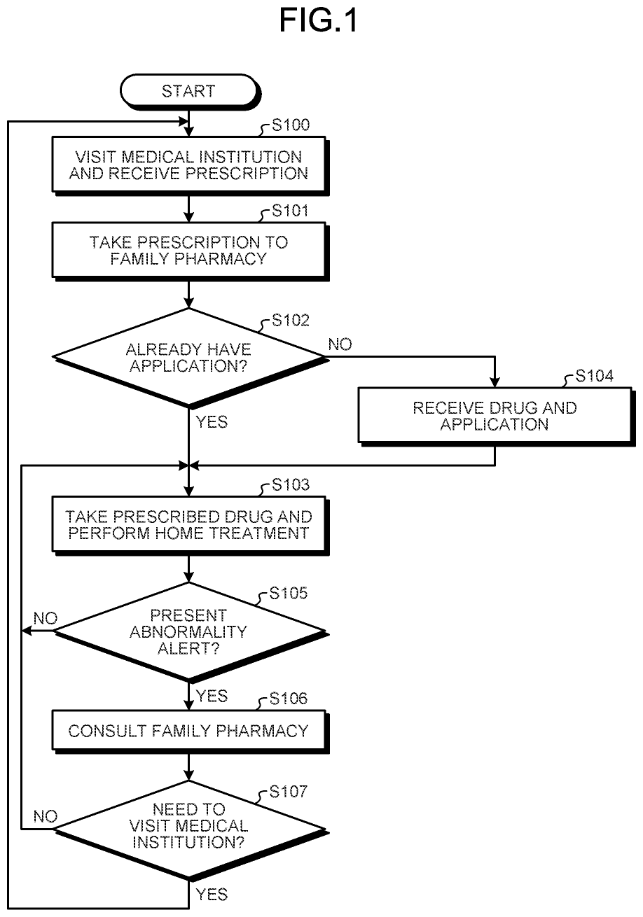

First, before explaining the present embodiment of the present disclosure created by the present inventors, the background leading to the creation of the present embodiment by the present inventors will be described with reference to FIG. 1. FIG. 1 is a flowchart illustrating a user's usage procedure in the treatment support system. As explained earlier, with the prospect of a shortage of doctors in the future, various treatment support systems have been developed to support treatment, and as one of the above treatment support systems, a treatment support system that determines whether a patient undergoing home treatment visits a medical institution can be exemplified. An example of the usage procedure for a user (patient) of such a treatment support system will be described below with reference to FIG. 1.

For example, a user visits a medical institution, receives a prescription from a doctor (step S100), and takes the received prescription to a family pharmacy (step S101). Next, depending on whether the user already has an application (or device) for using the treatment support system (step S102), if the user has the application (or device) (step S102: Yes), the user receives a prescription drug, and starts home treatment (for example, the user takes a prescription drug at home or the like) (step S103). On the other hand, if the user does not have the application (or device) (step S102: No), the user receives the above application (or device) and the prescription drug (step S104), and starts home treatment (step S103).

Next, a monitor device (not illustrated) (for example, a sphygmomanometer or the like) included in the treatment support system monitors a physical condition index (for example, blood pressure or the like) of a user who is undergoing home treatment. Then, in the treatment support system, the application presents an abnormality alert to the user when it is determined that the monitored physical condition index is abnormal. Therefore, depending on whether the abnormality alert is presented (step S105), specifically, when the abnormality alert is presented (step S105: Yes), the user consults the family pharmacy (step S106). When no abnormality alert is presented (step S105: No), the user returns to step S103 and continues home treatment.

Then, according to the determination by the pharmacist of the family pharmacy whether it is necessary to visit the medical institution (step S107), the procedure that the user is to perform next is determined. When it is determined that it is necessary to visit the medical institution (step S107: Yes), the user returns to step S100 and visits the medical institution. When it is determined that it is not necessary to visit the medical institution (step S107: No), the user returns to step S103 and continues home treatment.

According to such a treatment support system, the user can appropriately visit the medical institution when necessary, so that it is possible for the user to avoid an unnecessary visit to the medical institution or a visit to an emergency outpatient service, or the like. As a result, according to the treatment support system, the shortage of doctors can be alleviated.

Furthermore, the present inventors have made extensive studies in order to enhance the effectiveness of the above-mentioned treatment support system. In the study, the present inventors have noticed that the effectiveness of the treatment support system may be impaired when the abnormality of the physical condition index is detected by using a general-purpose threshold value. Here, the general-purpose threshold (general-purpose threshold value) is a reference value, recommended value or target value indicated in general treatment guidelines. In the above treatment support system, when the value of the monitored physical condition index (monitor value) exceeds or falls below the general-purpose threshold value, abnormality in the physical condition index is detected assuming that the user's body may be abnormal.

For example, even if the monitor value exceeds the general-purpose threshold value due to the medication status of the user's prescription drug, that is, even when it is clear that the result is not caused by an abnormality in the user's body, it will be detected as an abnormality in the method using the general-purpose threshold value described above. In such a case, even when it is not necessary to visit a medical institution, the user will visit the medical institution, resulting in an increase in the number of unnecessary visits to the medical institution or the number of emergency outpatient visits, or the like. Furthermore, since the number of abnormality alerts presented to the user increases, the psychological burden on the user increases, and the user may feel troublesome and may not visit a medical institution according to the abnormality alert. As described above, when the abnormality of the physical condition index is detected by using the general-purpose threshold value, the effectiveness of the treatment support system may be impaired.

Therefore, the present inventors have created a treatment support system (information processing system) according to the embodiment of the present disclosure that can properly determine whether to visit a medical institution by not only detecting the abnormality of the physical condition index using the general-purpose threshold value, but also evaluating the above physical condition index according to the user's situation. Specifically, in the present embodiment, when the cause is clear based on the user's situation (for example, the value of the physical condition index has increased due to not taking the prescription drug), or when it is determined that it is not abnormal compared to other users who have a situation similar to the user, the system only records the abnormality of the physical condition index and does not present the abnormality alert. Therefore, according to the present embodiment, it is possible to perform an abnormality alert according to the user's situation, in other words, personalized abnormality alerts. Therefore, while suppressing an increase in unnecessary abnormality alerts, it is possible to avoid an increase in the number of unnecessary visits to the medical institution or the number of emergency outpatient visits, or the like. In addition, according to the present embodiment, it is possible to avoid an increase in the psychological burden on the user. That is, according to the present embodiment, it is possible to provide a user or the like with a treatment support system having higher effectiveness.

In addition, the treatment support system according to the present embodiment has a configuration in which a family pharmacy or a pharmacist can offer the user's consultation before the user visits a medical institution, so that the burden on the doctor can be reduced and it is possible to enhance the safety and effectiveness of the medication treatment for the user. Furthermore, in the treatment support system according to the present embodiment, the monitored physical condition index values (monitor value) of the user are databased and stored in the server or the like, so that it facilitates information sharing between the user, the doctor, and the pharmacist and is helpful in advancing treatment more effectively. Hereinafter, details of such an embodiment according to the present disclosure will be sequentially described.

In the following description, the user means general users who use the treatment support system (the information processing system) according to the embodiment of the present disclosure, and more specifically, include patients who continue to receive home treatment while taking medication, their families, and medical professionals. Further, in the following description, the physical condition indexes mean biometric information such as heart rate, pulse rate, blood pressure, blood flow, respiratory volume, calories burned, brain wave, body temperature, skin electrical resistance, sweating, muscle activity, sleep time, calorie intake, the amount of exercise (for example, the number of steps) and the like, and may also include biometric information such as the color of the user's eyeballs and the presence or absence of bleeding.

### 2. FIRST EMBODIMENT

### 2.1 Schematic Configuration of Treatment Support System 1

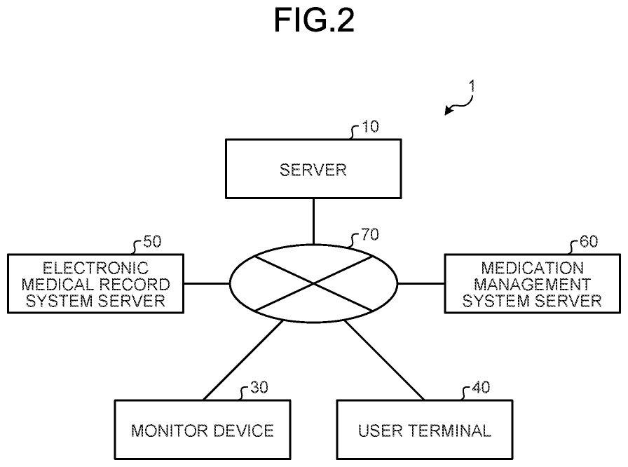

First, with reference to FIG. 2, a schematic configuration of a treatment support system (information processing system) 1 according to the first embodiment of the present disclosure will be described. FIG. 2 is a system diagram illustrating a schematic functional configuration of a treatment support system 1 according to the first embodiment of the present disclosure.

As illustrated in FIG. 2, the treatment support system 1 according to the present embodiment includes a server (information processing device) 10, a monitor device 30, a user terminal 40, an electronic medical record system server (medical record management device) 50, and a medication management system server (medication management device) 60, which are communicably connected to each other via a network 70. Specifically, the server 10, the monitor device 30, the user terminal 40, the electronic medical record system server 50, and the medication management system server 60 are connected to the network 70 via a base station or the like (not illustrated) (for example, a mobile phone base station, an access point of a wireless local area network (LAN), and the like). The communication method used in the network 70 may be any method regardless of wired or wireless method (for example, WiFi (registered trademark), Bluetooth (registered trademark), etc.), but it is desirable to use a communication method that can maintain a stable operation. Further, the number of the monitor devices 30 and the number of the user terminals 40 included in the treatment support system 1 are not limited to one each as illustrated in FIG. 2, and may be plural. The outline of each device included in the treatment support system 1 according to the present embodiment will be described below.

(Server 10)

The server 10 is composed of, for example, a computer (information processing device) and the like. The server 10 can evaluate, for example, the physical condition index (monitor value) of the user monitored by the monitor device 30 to be described later, and output the information obtained by the evaluation to another device (for example, the user terminal 40 to be described later) or the like. The details of the server 10 will be described later.

(Monitor Device 30)

The monitor device 30 is a device that monitors one or more physical condition indexes of the user. Specifically, the monitor device 30 includes various biometric information sensors such as, for example, a heartbeat sensor, a pulse sensor, a blood flow sensor (including a blood pressure sensor), a respiratory sensor (including a calorie consumption meter based on the amount of breathing), an electroencephalogram sensor, a skin temperature sensor, and a skin conductivity sensor, a sweating sensor, and a myoelectric sensor, and can acquire sensing data related to a user's physical condition indexes. Further, the monitor device 30 can be a wearable device that can be mounted on part of the user's body (earlobe, neck, arm, wrist, ankle, etc.). Further, the monitor device 30 may be incorporated in, for example, a general-purpose personal computer (PC), a tablet terminal, a game machine, a mobile phone such as a smartphone, an in-vehicle device (car navigation device, seat, etc.) and the like. The details of the monitor device 30 will be described later.

(User Terminal 40)

The user terminal 40 is a terminal for use by the user or the medical professional, and is further installed in the vicinity of the user or the medical professional to output the information obtained by the server 10 to the user or the like. Further, the user terminal 40 can also receive the information input from the user or the medical professional to output the received information to the server 10. For example, the user terminal 40 can be a device such as a tablet PC, a smartphone, a mobile phone, a laptop PC, a notebook PC, or a head mounted display (HMD). Further, the user terminal 40 includes a display unit (not illustrated) that displays an image for the user and the medical professional, an input unit (not illustrated) that receives an input operation from the user and the medical professional, and a speaker (not illustrated) that outputs audio to the user and the medical professional. In the present embodiment, the user terminal 40 may be provided with various biometric information sensors included in the monitor device 30 described above.

(Electronic Medical Record System Server 50)

The electronic medical record system server 50 is configured by, for example, a computer and the like, and manages the information on the electronic medical record for the treatment of the user created by the medical professional. In the present embodiment, the above-mentioned server 10 can use the data of the electronic medical record stored in the electronic medical record system server 50.

(Medication Management System Server 60)

The medication management system server 60 is configured by, for example, a computer and the like, manages the presence or absence of medication based on the user's medication declaration, and guides the user to take the drug according to the medication procedure determined by the medical professional (FOR example, at the time of taking the drug, an alert prompting the user to take the drug can be presented). In the present embodiment, the above-mentioned server 10 can use the data of the medication information (user's medication status) stored in the medication management system server 60.

The treatment support system 1 according to the present embodiment may include, for example, another communication device such as a relay device that transmits information from the monitor device 30 to the server 10. Further, in the present embodiment, two or all of the server 10, the monitor device 30, and the user terminal 40 may be an integrated device, that is, they may not be each realized by a single device. In addition, in the present embodiment, the server 10, the monitor device 30, and the user terminal 40 are connected to each other via various wired or wireless networks 70, and may be realized by a plurality of devices that cooperate with each other.

### 2.2 Detailed Configuration of Server 10

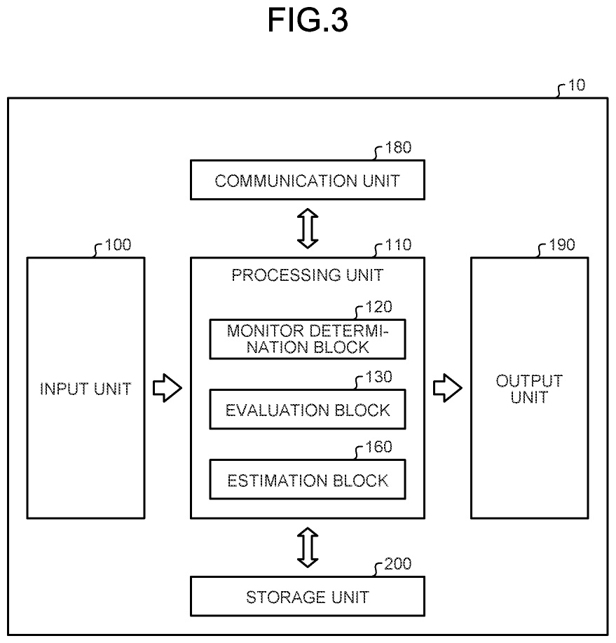

As described above, the server 10 according to the present embodiment can evaluate the physical condition index (monitor value) of the user monitored by the monitor device 30, and output the information obtained by the evaluation to another device or the like. The detailed configuration of the server 10 will be described with reference to FIG. 3. FIG. 3 is a diagram illustrating a functional configuration of the server 10 according to the present embodiment. As illustrated in FIG. 3, the server 10 can mainly include an input unit 100, a processing unit 110, a communication unit 180, an output unit 190, and a storage unit 200. Hereinafter, each functional block of the server 10 will be described in sequence.

(Input Unit 100)

The input unit 100 receives input operations of data and a command from the user and the medical professional to the server 10, or input operations of data and a command from the administrator of the server 10 to output the input information to the processing unit 110 to be described later. More specifically, the input unit 100 is realized by a touch panel, a keyboard, or the like. When the input unit 100 is a touch panel, the input unit 100 may be combined with an image display device (not illustrated).

(Processing Unit 110)

The processing unit 110 is provided in the server 10 and can control each functional block of the server 10. The processing unit 110 is realized by hardware such as a central processing unit (CPU), a read only memory (ROM), and a random access memory (RAM), for example. In detail, as illustrated in FIG. 3, the processing unit 110 can be divided into three main functional blocks of a monitor determination block 120, an evaluation block 130, and an estimation block 160. Details of these functional blocks will be described later for each block.

(Communication Unit 180)

The communication unit 180 is provided in the server 10 and can transmit and receive information to and from an external device such as the monitor device 30 and the user terminal 40. The communication unit 180 is realized by a communication device such as a communication antenna, a transmission/reception circuit, and a port.

(Output Unit 190)

The output unit 190 it is composed of, for example, a display, a speaker, a video output terminal, an audio output terminal, etc. and outputs various information obtained by the above-mentioned processing unit 110 to users and medical professionals by an image or sound, etc. Specifically, the output unit 190 can output predetermined information according to the evaluation result obtained by the processing unit 110 and the type of the physical state information index processed by the processing unit 110.

(Storage Unit 200)

The storage unit 200 is provided in the server 10 and stores a program or the like for the processing unit 110 described above to execute various kinds of processing, and information obtained by the processing. More specifically, the storage unit 200 can store the history of the physical condition indexes acquired from a plurality of users. The storage unit 200 is realized by, for example, a recording device such as a hard disk drive (HDD), a non-volatile memory, or the like.

### 2.3 Detailed Configuration of Monitor Determination Block 120

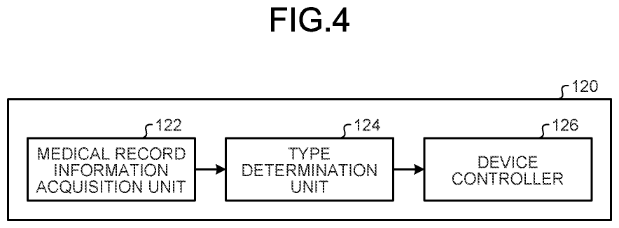

As described above, the processing unit 110 can be divided into three main functional blocks of the monitor determination block 120, the evaluation block 130, and the estimation block 160. First, with reference to FIG. 4, each functional unit of the monitor determination block 120 of the processing unit 110 will be described in sequence. FIG. 4 is a diagram illustrating a functional configuration of the monitor determination block 120 according to the present embodiment. Specifically, as illustrated in FIG. 4, the monitor determination block 120 of the processing unit 110 mainly includes a medical record information acquisition unit 122, a type determination unit 124, and a device controller 126. Hereinafter, each functional unit of the monitor determination block 120 will be described in sequence.

(Medical Record Information Acquisition Unit 122)

The medical record information acquisition unit 122 acquires electronic medical record information from the electronic medical record system server 50 that manages the medical record created by the medical professional, to output the information to the type determination unit 124, which will be described later. For example, the information on the electronic medical record can include the name of the disease being treated of the user, the medical condition, the treatment start date, the treatment target (for example, the value of the physical condition index when the patient is completely cured (100%), etc.), the items of the physical condition indexes to be monitored (monitor items) (for example, blood pressure, etc.), the management items (for example, meal management, etc.), the information on the drug being taken (brand name, number of doses, action, side effects, medication precautions, etc.), and the like. Further, the electronic medical record information can include user attribute information (gender, age, height, weight, etc.). That is, the treatment support system 1 according to the present embodiment can cooperate with the electronic medical record.

(Type Determination Unit 124)

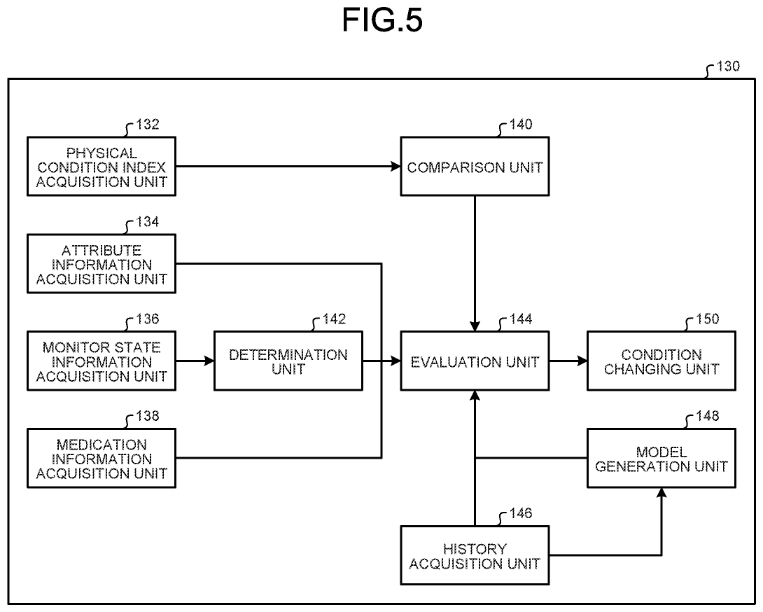

The type determination unit 124 determines the type (monitor item) of the physical condition index acquired by the physical condition index acquisition unit 132 (see FIG. 5), which will be described later, based on the electronic medical record information from the medical record information acquisition unit 122 to output the determined type to the device controller 126, which will be described later. For example, when the electronic medical record information includes blood pressure as an item of a physical condition index to be monitored, the type determination unit 124 determines blood pressure as a monitor item. In the present embodiment, the type determination unit 124 is not limited to determining the monitor item based on the electronic medical record information, and may determine the item based on the input information from the user or the medical professional, or may determine the type by automatically extracting the type from a medical database (not illustrated) according to the name of the disease.

Further, the type determination unit 124 automatically extracts a general-purpose threshold value (first threshold value) and monitoring conditions (time to monitor, user's posture when monitoring, user's behavior before monitoring, mounting state, etc.) from medical database (not shown) based on the determined monitor item, and outputs them to the device controller 126, which will be described later, and the comparison unit 140 of the evaluation block 130, which will be described later. In the present embodiment, the automatically extracted general-purpose threshold value and monitoring conditions may be modified or the condition may be added by an input operation from the user or the medical professional.

(Device Controller 126)

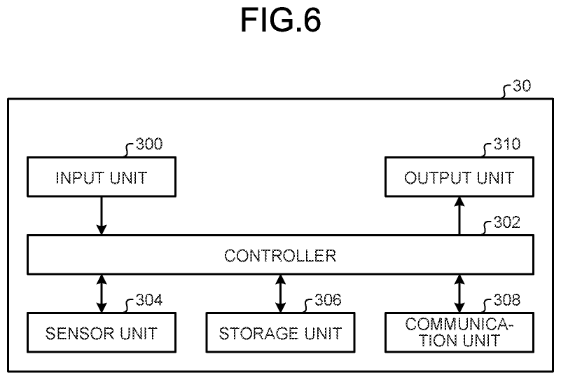

The device controller 126 generates control information that controls a corresponding sensor unit 304 (see FIG. 6) according to the monitor items and monitoring conditions determined by the type determination unit 124, and transmits the information to the monitor device 30 via the communication unit 180. For example, the device controller 126 controls the sphygmomanometer so as to pair with the sphygmomanometer of the sensor unit 304 and monitor the blood pressure as a physical condition index according to the generated control information.

### 2.4 Detailed Configuration of Evaluation Block 130

Next, with reference to FIG. 5, each functional unit of the evaluation block 130 of the processing unit 110 will be sequentially described. FIG. 5 is a diagram illustrating a functional configuration of the evaluation block 130 according to the present embodiment. The detailed configuration of the estimation block 160 of the processing unit 110 will be described in the second embodiment to be described later. Specifically, as illustrated in FIG. 5, the evaluation block 130 of the processing unit 110 includes a physical condition index acquisition unit 132, an attribute information acquisition unit 134, a monitor state information acquisition unit 136, and a medication information acquisition unit 138. Further, the evaluation block 130 includes a comparison unit 140, a determination unit 142, an evaluation unit 144, a history acquisition unit 146, a model generation unit 148, and a condition changing unit 150. Hereinafter, each functional unit of the evaluation block 130 will be described in sequence.

(Physical Condition Index Acquisition Unit 132)

The physical condition index acquisition unit 132 acquires a physical condition index (monitor value) from the monitor device 30 that monitors one or more physical condition indexes of the user, to output the physical condition index (monitor value) to the comparison unit 140 to be described later.

(Attribute Information Acquisition Unit 134)

The attribute information acquisition unit 134 acquires the attribute information on the user from the above-mentioned medical record information acquisition unit 122 or the input operation from the user or the medical professional, to output the attribute information to the evaluation unit 144 to be described later. Specifically, the attribute information acquisition unit 134 acquires attribute information such as the user's age, gender, height, weight, and the user's daily schedule (for example, wake-up time, sleep time, activity amount, meal time, meal content, etc.). The acquired attribute information may be associated with the monitored physical condition index (monitor value) and stored in the storage unit 200.

(Monitor State Information Acquisition Unit 136)

The monitor state information acquisition unit 136 acquires sensing data indicating the time when the physical condition index (monitor value) is monitored, the mounting state of the monitor device 30, or the posture or activity state of the user from the monitor device 30, and outputs them to the determination unit 142 to be described later.

(Medication Information Acquisition Unit 138)

The medication information acquisition unit 138 acquires the user's medication information (presence or absence of medication and medication time) from the medication management system server 60 that manages medication based on the user's medication declaration, to output the information to the evaluation unit 144 to be described later. That is, the treatment support system 1 according to the present embodiment can cooperate with the medication management system.

(Comparison Unit 140)

The comparison unit 140 compares the monitored physical condition index (monitor value) acquired from the physical condition index acquisition unit 132 with a general-purpose threshold value set in advance for the type of the physical condition index, to output the comparison result to the evaluation unit 144 to be described later.

(Determination Unit 142)

The determination unit 142 determines whether the monitor value is monitored by the monitor device 30 under predetermined monitoring conditions based on the sensing data from the monitor state information acquisition unit 136, the user's schedule information from the attribute information acquisition unit 134, and the like. For example, the determination unit 142 can determine whether the value is monitored under the predetermined monitoring condition based on the sensing data indicating the time when the physical condition index was monitored, the mounting state of the monitor device 30, or the posture or activity state of the user. Further, the determination unit 142 outputs the determination result to the evaluation unit 144, which will be described later.

(Evaluation Unit 144)

The evaluation unit 144 compares the monitor value (monitored physical condition index) with the personalization threshold value (details will be described later) according to the comparison result by the comparison unit 140 (for example, when the monitor value exceeds the general-purpose threshold value in the comparison unit 140) to evaluate the monitor value. Specifically, the evaluation unit 144 compares the monitor value with the history of the physical condition index of the user or other users selected based on at least one of the attribute information and the medication information to evaluate the monitor value. For example, when the monitor value deviates from the distribution of the physical condition index of other users, the evaluation unit 144 evaluates (detects) that the monitor value is abnormal.

In addition, the evaluation unit 144 may compare the monitor value with the prediction value derived from the history of the user's physical condition index. Further, the evaluation unit 144 calculates a difference between the monitor value and the prediction value, and if the calculated difference exceeds a preset threshold value (second threshold value), the evaluation unit 144 may evaluate the difference as abnormal.

Further, the evaluation unit 144 may evaluate the monitor value by referring to the result of a determination by the determination unit 142 (whether the physical condition index is monitored by the monitor device 30 under predetermined monitoring conditions). In addition, the evaluation unit 144 may evaluate the monitor value by referring to the user's medication information (presence or absence of medication) from the medication information acquisition unit 138. Then, the evaluation unit 144 outputs the evaluation result to the output unit 190 and the condition changing unit 150 to be described later. The details of the evaluation method by the evaluation unit 144 will be described later.

(History Acquisition Unit 146)

The history acquisition unit 146 acquires the history of the user's physical condition index or the history of the physical condition index of other users having attribute information similar to the user's attribute information from the storage unit 200, and outputs it to the evaluation unit 144 and the model generation unit 148.

(Model Generation Unit 148)

The model generation unit 148 can generate a model for the prediction value or calculate the prediction value based on the history of the user's physical condition index. Specifically, the model generation unit 148 can generate (estimate) an autoregressive model from the history of the user's physical condition index and calculate a prediction value based on the autoregressive model. Further, the model generation unit 148 can output the calculated prediction value to the evaluation unit 144 described above. The details of model generation and prediction value calculation by the model generation unit 148 will be described later.

(Condition Changing Unit 150)

The condition changing unit 150 dynamically changes (updates) the monitoring condition (predetermined measurement condition) for monitoring the user's physical condition index according to the evaluation of the monitor value by the evaluation unit 144. Here, the monitoring condition means a condition such as a time, an activity state of the user (after exercise, after eating, before sleeping, etc.), and a posture at the time of monitoring. The condition changing unit 150 outputs the updated monitoring condition to the device controller 126 described above.

### 2.5 Detailed Configuration of Monitor Device 30

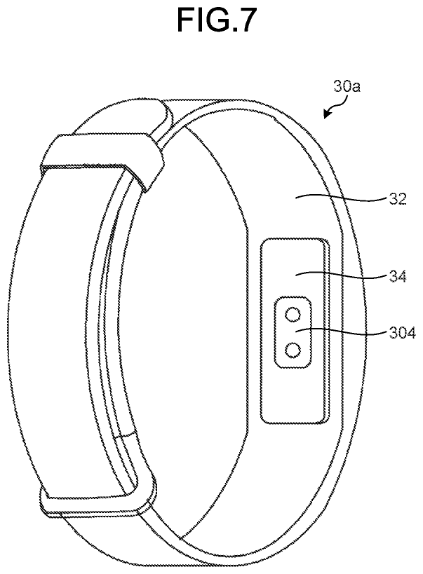

Next, the detailed configuration of the monitor device 30 according to the present embodiment will be described with reference to FIGS. 6 and 7. FIG. 6 is a diagram illustrating a functional configuration of the monitor device 30 according to the present embodiment, and FIG. 7 is a diagram illustrating an external example of a monitor device 30a according to the present embodiment. As described above, the monitor device 30 according to the present embodiment is a device that monitors one or more physical condition indexes of the user. As illustrated in FIG. 6, the monitor device 30 mainly includes an input unit 300, a controller 302, a sensor unit 304, a storage unit 306, a communication unit 308, and an output unit 310. Hereinafter, each functional unit of the monitor device 30 will be described in sequence.

(Input Unit 300)

The input unit 300 receives input of data and commands from the user to the monitor device 30, to output the information input by the received input operation to the controller 302 to be described later. More specifically, the input unit 300 is realized by a keyboard, a touch panel, buttons, a microphone, or the like.

(Controller 302)

The controller 302 is provided in the monitor device 30 and controls each functional unit of the monitor device 30. The controller 302 is realized by hardware such as a CPU, a ROM, and a RAM, for example. Some of the functions of the controller 302 may be provided by the server 10.

(Sensor Unit 304)

The sensor unit 304 can monitor at least one physical condition index related to the user to transmit the acquired physical condition index (monitor value) to the server 10 via the communication unit 308 to be described later. As described above, the sensor unit 304 can include various biometric information sensors such as a heartbeat sensor, a pulse sensor, a blood flow sensor, a respiratory sensor, an electroencephalogram sensor, a skin temperature sensor, a skin conductivity sensor, a sweating sensor, and a myoelectric sensor and acquire sensing data related to a user's physical condition index. For example, when the sensor unit 304 includes a plurality of sensors, the sensor unit 304 may be separated into a plurality of parts or may be separated from the monitor device 30.

For example, a heartbeat sensor is a sensor that detects a heartbeat, which is a beat in the user's heart. In addition, the pulse sensor is a sensor that detects the pulse, which is the pulse of the artery that appears on the surface of the body, and the like, due to the change in pressure on the inner wall of the artery caused by the blood being sent to the whole body through the artery by the beat (heartbeat) in the heart. Further, the blood flow sensor is, for example, a sensor that radiates infrared rays or the like to the body and detects blood flow, pulse, and heart rate based on the absorption rate or reflectance of light or its change. Further, the heartbeat sensor or pulse sensor may be an imaging device that images the user's skin, and in this case, detects the pulse and the heartbeat based on the change in the light reflectance in the skin obtained from the image of the user's skin. For example, the respiratory sensor can be a respiratory flow rate sensor that detects a change in respiratory volume. The electroencephalogram sensor is a sensor that detects an electroencephalogram by attaching a plurality of electrodes to the user's scalp and extracting periodic waves by removing noise from fluctuations in the measured potential difference between the electrodes. The skin temperature sensor is a sensor that detects the surface body temperature of the user, and the skin conductivity sensor is a sensor that detects the electrical resistance of the skin of the user. The sweating sensor is a sensor that is attached to the user's skin and detects a voltage or resistance, between two points on the skin, that changes due to sweating. In addition, the myoelectric sensor is a sensor that quantitatively detects the amount of muscle activity of muscles by measuring the myoelectric potential by an electrical signal generated in the muscle fibers when the muscles of the arm or the like contract by a plurality of electrodes attached to the user's arm or the like and propagates to the body surface.

Further, the sensor unit 304 may be realized by an imaging device that captures images of the user's eyes, oral cavity, around the nostrils, and the whole body as an imaging range. The color of the user's eyeball and the color of the user's gums, the presence or absence of nosebleed, etc. may be detected by the imaging device. More specifically, for example, the presence or absence of jaundice may be detected by the color of the white eye portion of the user's eyeball captured by the imaging device. Further, the sensor unit 304 may be a microphone that collects the user's voice, and, for example, may extract a word such as “blood has come out” from the user's voice and detect the presence or absence of bleeding.

Further, the sensor unit 304 may include a position sensor that detects a position of the user, a motion sensor that detects a movement of the user, and the like.

The position sensor is a sensor that is attached or carried by the user to detect the position of the user, and specifically can be a global navigation satellite system (GNSS) receiver or the like. In this case, the position sensor can generate sensing data indicating the latitude and longitude of the user's current location based on the signal from the GNSS satellite. Further, in the present embodiment, for example, it is possible to detect the relative positional relationship of the user from a radio frequency identification (RFID), a Wi-Fi access point, radio base station information, and the like, so that it is possible to use such a communication device as the position sensor. In the present embodiment, by detecting the position of the user, it is possible to detect the behavior of the user (for example, detecting that the user is sleeping because the user is in the bedroom).

Further, the motion sensor acquires sensing data indicating the state (amount of exercise, etc.) of each movement element performed by each part of the user's body by, for example, being attached to part of the user's body or a tool used by the user. For example, the motion sensor is realized by one or more sensor devices such as a 3-axis acceleration sensor, a 3-axis angular velocity sensor, a gyro sensor, a geomagnetic sensor, a position sensor, a vibration sensor, and a bending sensor, and sensor devices such as those described above detect changes in acceleration, angular velocity, and the like given by motion elements to generate a plurality of sensing data indicating the detected changes. Further, the sensor device as described above can function as a posture sensor that detects not only the state of each motion element performed by each part of the user's body but also the posture of the user. For example, the sensing data acquired by the motion sensor can be used to detect the posture of the user when the physical condition index is monitored, or to detect whether the user is sleeping.

Further, in the present embodiment, the motion sensor may be an imaging device that images a user. Specifically, a marker consisting of a light emitting diode (LED) or the like is attached to the user's joint or finger, and the movement of the marker is captured by a high-speed camera to quantitatively detect the position and movement of the user's joint.

Further, the sensor unit 304 may include a sensor that detects the mounting state of the sensor unit 304 and, for example, can include a pressure sensor that detects that the sensor unit 304 is correctly mounted on part of the user's body.

(Storage Unit 306)

The storage unit 306 is provided in the monitor device 30 and stores programs, information, and the like for the controller 302 described above to execute various kinds of processing, and information (for example, monitor values, etc.) obtained by the processing. The storage unit 306 is realized by, for example, a non-volatile memory such as a flash memory.

(Communication Unit 308)

The communication unit 308 is provided in the monitor device 30 and can send and receive information to and from an external device such as the server 10. In other words, the communication unit 308 can be said to be a communication interface having a function of transmitting and receiving data. The communication unit 308 is realized by a communication device such as a communication antenna, a transmission/reception circuit, and a port.

(Output Unit 310)

The output unit 310 is a device for presenting information to a user or the like, to output various types of information to the user by means of an image, sound, light, vibration, or the like. More specifically, the output unit 310 can display the information provided by the server 10 on the screen. The output unit 310 is realized by a display, a speaker, earphones, a light emitting element (for example, an LED), a vibration module, or the like. Some of the functions of the output unit 310 may be provided by the user terminal 40.

The monitor device 30 can be a wearable device such as a device that can be attached to part of the user's body (earlobe, neck, arm, wrist, ankle, etc.) or an implant device (implant terminal) inserted into the user's body. More specifically, the monitor device 30 can be a wearable device of various types such as HMD type, eyeglass type, ear device type, anklet type, bracelet (wristband) type, collar type, eyewear type, pad type, batch type, and clothing type. Further, the monitor device 30 may be incorporated in, for example, a general-purpose PC, a tablet terminal, a game machine, a mobile phone such as a smartphone, an in-vehicle device (car navigation device, a seat, etc.) and the like.

For example, as illustrated in FIG. 7, the monitor device 30 may be a bracelet-shaped monitor device 30a attached to the user's wrist. In detail, as illustrated in FIG. 7, the monitor device 30a has a belt-shaped band portion 32 and a control unit 34. Since the band portion 32 is worn so as to be wrapped around the user's wrist, for example, the band portion 32 is formed of a material such as soft silicone gel so as to form a ring shape according to the shape of the wrist. Further, the control unit 34 is a portion provided with the above-mentioned sensor unit 304, the controller 302, and the like. Further, the sensor unit 304 is provided at a position where the monitor device 30a is in contact with or faces the user's body when the monitor device 30a is attached to part of the user's body.

### 2.6 Information Processing Method

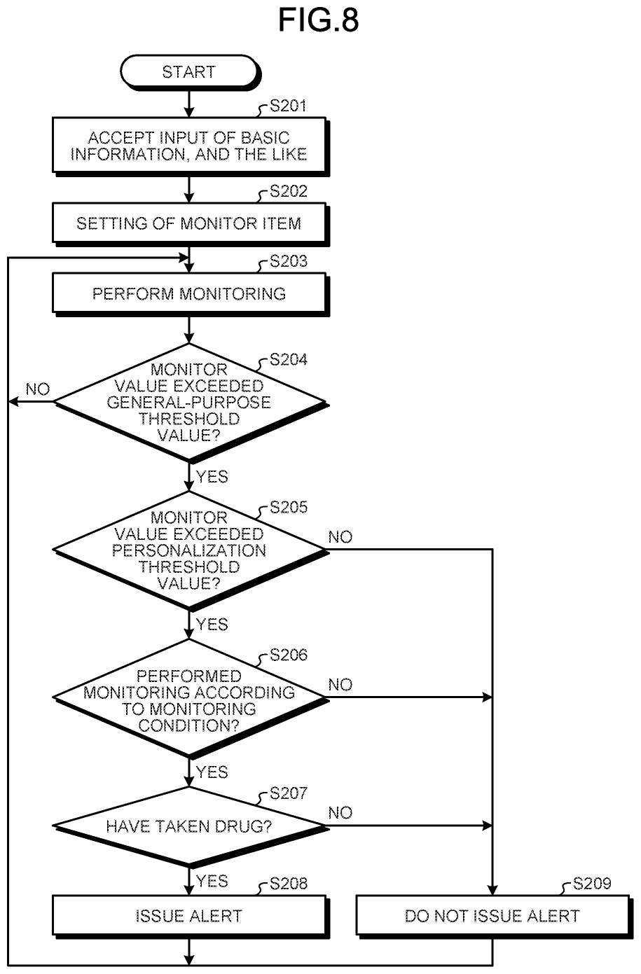

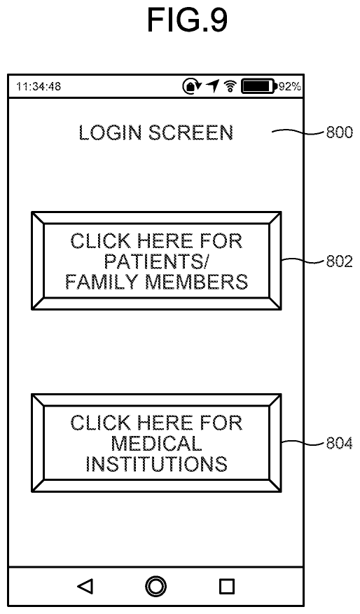

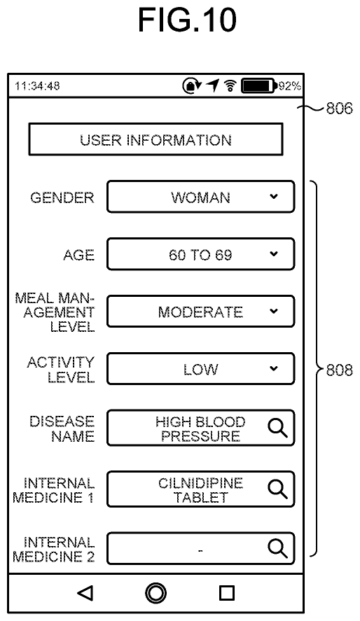

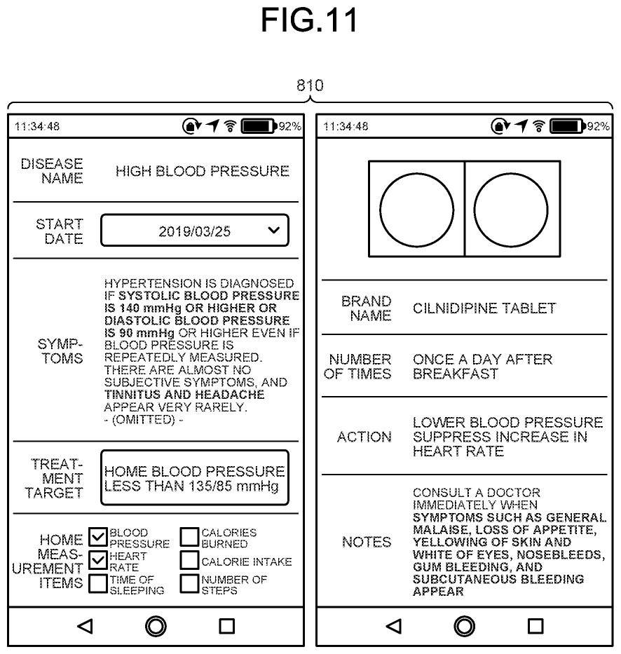

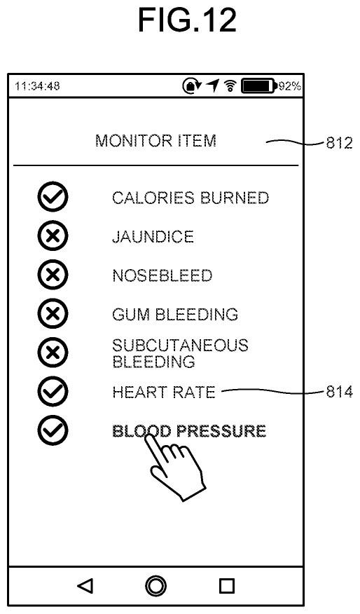

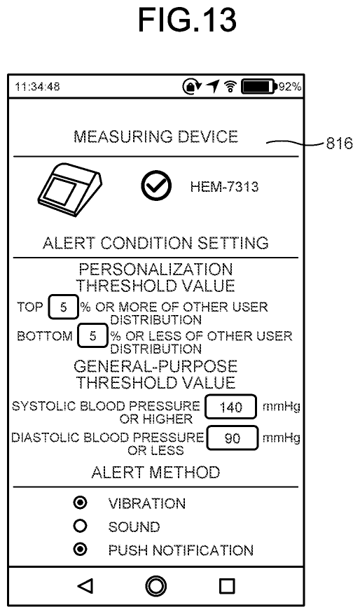

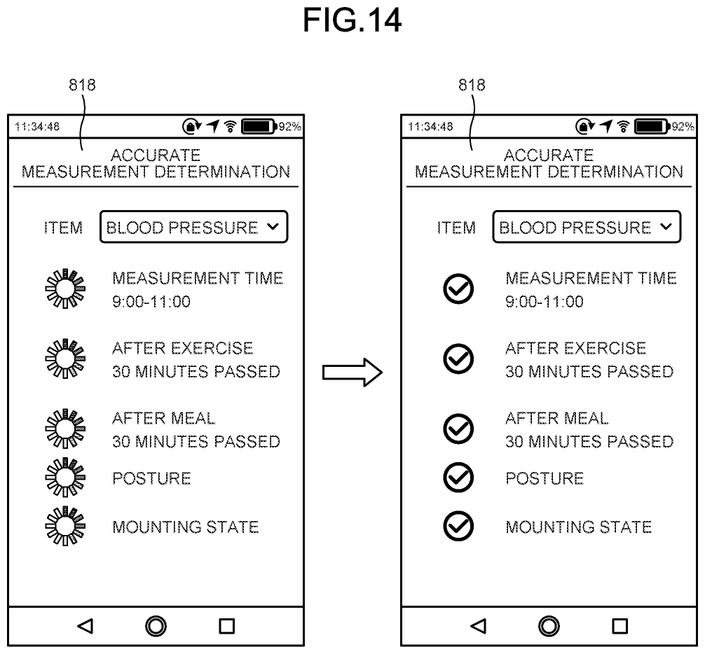

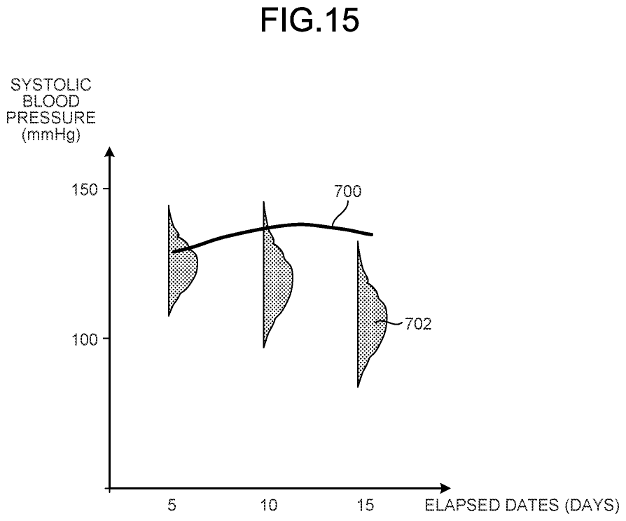

Next, the information processing method according to the first embodiment of the present disclosure will be described with reference to FIGS. 8 to 17. FIG. 8 is a flowchart illustrating an information processing method according to the present embodiment. FIG. 9 is an explanatory diagram illustrating an example of a login screen 800 according to the present embodiment, FIG. 10 is an explanatory diagram illustrating an example of an input screen 806 according to the present embodiment, and FIG. 11 is an explanatory diagram illustrating an example of a management screen 810 according to the present embodiment. FIG. 12 is an explanatory diagram illustrating an example of a monitor item setting screen 812 according to the present embodiment, FIG. 13 is an explanatory diagram illustrating an example of a monitor device management screen 816 according to the present embodiment, and FIG. 14 is an explanatory diagram illustrates an example of the determination screen 818 according to the present embodiment. Further, FIGS. 15 to 17 are explanatory views for explaining the evaluation method according to the present embodiment.

As illustrated in FIG. 8, the information processing method according to the present embodiment can mainly include steps from step S201 to step S209. The steps from step S201 to step S209 correspond to step S105 in FIG. 1. The details of each of these steps according to the present embodiment will be described below. Further, in the information processing method described below, steps S203 to S209 are repeatedly performed until the user is completely cured.

First, the server 10 accepts the input of basic information (step S201). Specifically, the server 10 acquires from electronic medical record information from the electronic medical record system server 50, and input operations from the user or the medical professional, the user attribute information, the name of the disease being treated of the user, the medical condition, the treatment start date, the treatment target, the items of the physical condition indexes to be monitored (monitor items), the management items, the information on the drug being taken, and the like.

Specifically, when the above information is input to the server 10 by an input operation from the user (including the user's family) or the medical professional, for example, the operation is performed on the login screen 800 as illustrated in FIG. 9 displayed on the input unit (not illustrated) of the user terminal 40. The login screen 800 includes a button 802 for transitioning to a mode in which a user and a user's family perform an input operation, and a button 804 for transitioning to a mode in which a medical professional performs an input operation. The login screen 800 illustrated in FIG. 9 is merely an example, and the present embodiment is not limited to such a screen, and may further include other displays and the like. Further, in the present embodiment, in order to protect the personal information on the user during the input operation, it is preferable to perform personal authentication in which the input user or the medical professional who performs an input operation is face-authenticated, fingerprint-authenticated, authenticated by the license card information on the medically qualified person, and the like.

For example, the user's attribute information (age, gender, height, weight, user's daily schedule (wake-up time, sleep time, activity amount, meal time, meal content, etc.), etc.) can be acquired by the user or the medical professional performing an input operation on the input screen 806 as illustrated in FIG. 10 displayed on the input unit (not illustrated) of the user terminal 40. For example, the input screen 806 is displayed when the mode is changed as a result of operating the button 802 described above. The input screen 806 includes a plurality of input fields 808 for the user and the user's family to input each item of the attribute information. The input screen 806 illustrated in FIG. 10 is merely an example, and the present embodiment is not limited to such a screen, and may further include other displays and the like. Further, the above-mentioned user attribute information may be acquired by, for example, being extracted from the information on the electronic medical record.

For example, the name of the disease being treated of the user, the medical condition, the treatment start date, the treatment target, the items of the physical condition indexes to be monitored (for example, blood pressure, etc.), the management items (for example, meal management), the information on the drug being taken (brand name, number of doses, action, side effects, medication precautions, etc.), and the like are acquired by being extracted from the information on the electronic medical record based on the patient number of the user. The extracted information can be presented to the user or the like on the management screen 810 as illustrated in FIG. 11 displayed on the user terminal 40. The management screen 810 illustrated in FIG. 11 is merely an example, and the present embodiment is not limited to such a screen, and may further include other displays and the like.

Next, returning to FIG. 8 and continuing the explanation, the server 10 sets the type (monitor item) of the physical condition index to be monitored (step S202). Specifically, the server 10 sets the above monitor item based on electronic medical record information, input information from the user or the medical professional, or a medical database (not illustrated). For example, the user or the like can set a monitor item by performing an input operation on the monitor item setting screen 812, as illustrated in FIG. 12 displayed on the user terminal 40, which includes a plurality of monitor items 814. The monitor item setting screen 812 illustrated in FIG. 12 is merely an example, and the present embodiment is not limited to such a screen, and may further include other displays and the like.

Further, after the monitor items are set, for example, the user terminal 40 displays the monitor device management screen 816 as illustrated in FIG. 13. The monitor device management screen 816 displays information (for example, device name, etc.) about the measuring device (biometric information sensor) paired (communicably connected) with the server 10 corresponding to the set monitor item. At this time, when the measuring device corresponding to the monitor item is not paired with the server 10, the server 10 may present to the user or the like a display to guide the user to start or to pair the measuring device. In addition, the monitor device management screen 816 can display the conditions for presenting an abnormality alert (general-purpose threshold value, personalization threshold value (details will be described later)) and the presentation method, and ask the user and the medical professional to perform validation and correction. The monitor device management screen 816 illustrated in FIG. 13 is merely an example, and the present embodiment is not limited to such a screen, and may further include other displays and the like.

Returning to FIG. 8 and continuing the description, the server 10 monitors the user's physical condition index according to the monitor item set in step S202 (step S203). At this time, for example, the user terminal 40 displays the determination screen 818 as illustrated on the left side of FIG. 14. The determination screen 818 displays predetermined monitoring conditions (monitor time, user status before measurement, posture, mounting state of the sensor unit 304, etc.) based on electronic medical record information, input information from a user or a medical professional, or a medical database (not shown). Further, the server 10 determines based on the sensing data from the sensor unit 304 and the like whether each item of the monitoring conditions is satisfied, and displays the determination result on the determination screen 818 as illustrated on the right side of FIG. 14. The determination screen 818 illustrated in FIG. 14 is merely an example, and the present embodiment is not limited to such a screen, and may further include other displays and the like.

Further, the server 10 stores the monitored physical condition index (monitor value) of the user in the storage unit 200 in association with information such as the user's attribute information and measurement items. In the present embodiment, by doing so, the history of the monitored physical condition index of the user is stored as a database, so that it facilitates information sharing between the user, the doctor, and the pharmacist and is helpful in advancing treatment more effectively.

Returning to FIG. 8 and continuing the description, the server 10 determines whether the monitor value exceeds the general-purpose threshold value (step S204). When it is determined that the monitor value exceeds the general-purpose threshold value (step S204: Yes), the server 10 advances to the processing in step S205. On the other hand, when it is determined that the monitor value does not exceed the general-purpose threshold value (step S204: NO), the server 10 returns to the processing in step S203 and continues monitoring.

Then, the server 10 determines whether the monitor value exceeds the personalization threshold value (step S205). When it is determined that the monitor value exceeds the personalization threshold value (step S205: Yes), the server 10 advances to the processing in step S206. On the other hand, when it is determined that the monitor value does not exceed the personalization threshold value (step S205: NO), the server 10 advances to the processing in step S209. In the present embodiment, the setting of the personalization threshold value can be changed according to the status of the history (data) of the user's physical condition index stored in the storage unit 200. The personalization threshold value will be described below.

—When the User's Physical Condition Index History is not Sufficient—

In the present embodiment, when the history (data) of the user's physical condition index stored in the storage unit 200 is not sufficient, the personalization threshold value is set using the history of the physical condition index of other users having attribute information or the like similar to the user's attribute information. For example, the server 10 acquires the distribution 702 of a physical condition index of other users, for each treatment elapsed date, which have attribute information similar to the user's attribute information as illustrated in FIG. 15, and at the same monitor time as the user's monitor value. Then, the server 10 sets the personalization threshold value based on the distribution 702 of the physical condition index of other users at the treatment elapsed date same as that of the monitor value. More specifically, for example, when the medical professional sets the range of the personalization threshold value in advance with the top 5% of the distribution 702 of the physical condition index of other users as the upper limit value, and the bottom 5% of the distribution 702 of the physical condition index of other users as the lower limit, the range of the personalization threshold value is in the range of the top 5% to the bottom 5% of the distribution 702 of the physical condition index of other users. Then, when a monitor value 700 is out of the range of the personalization threshold value, the server 10 detects an abnormality of the monitor value 700.

In the above method, the personalization threshold value is set by using the history of the physical condition index of other users having the attribute information similar to the attribute information on the user, but the present embodiment is not limited to this. For example, in the present embodiment, a feature point and a feature amount for the history of the physical condition index are extracted using machine learning by a recurrent neural network or the like, cluster classification is performed, and the history of the physical condition index of other users classified into the same cluster as the physical condition index of the user may be extracted as the data for generating the personalization threshold value. Here, the cluster refers to a group of data, having a similar tendency, that can be estimated using the same model.

—When the User's Physical Condition Index has a Sufficient History—

In the present embodiment, when the history (data) of the user's physical condition index stored in the storage unit 200 is sufficient, the personalization threshold value is set by using the history of the user's physical condition index.

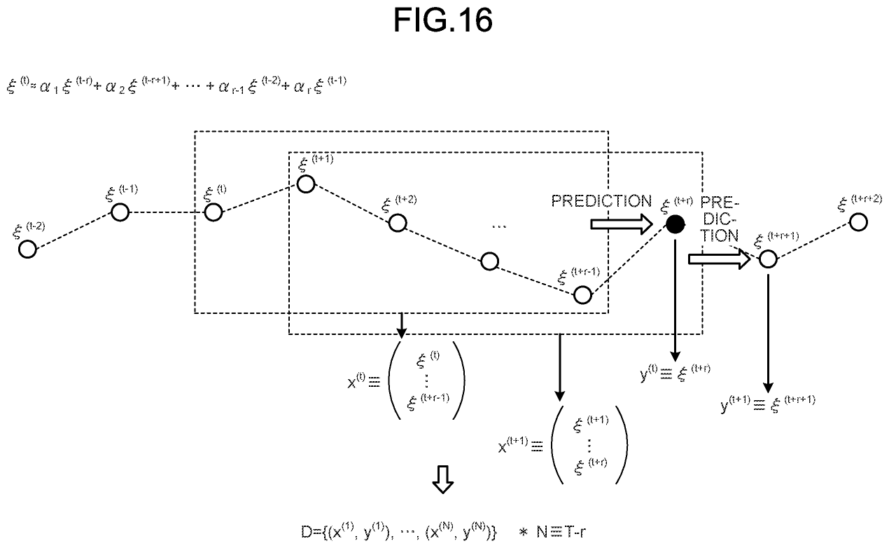

For example, the server 10 uses the history of the user's physical condition index stored in the storage unit 200 as training data to generate an autoregressive model from the training data. As illustrated in the upper equation in FIG. 16, the autoregressive model is a model in which ξ^{(t)} at a certain time t can be expressed by a set of past data ξ^{(t-r)} with respect to the certain time t and a set of coefficient parameters αᵣ of each data ξ^{(t-r)}. As illustrated in the lower figure in FIG. 16, ξ^{(t)} at the time t, that is, a value of the physical condition index at the same time as the physical condition index (monitor value) of the monitored user is predicted from the set of past data ξ^{(t-r)}, that is, the history of the user's physical condition index by the same approach as linear regression based on the model.

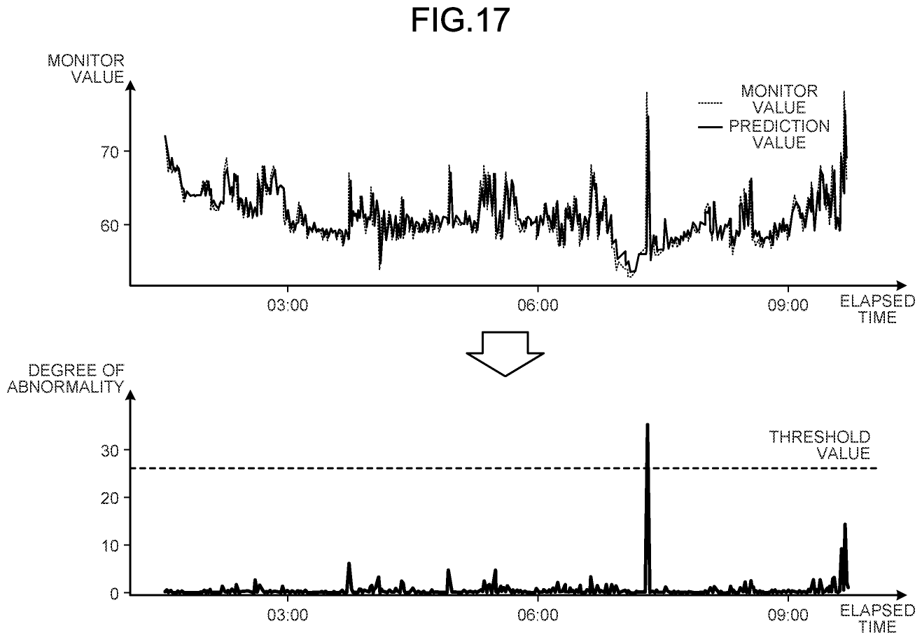

Further, the server 10 can compare the monitor value with the prediction value (personalization threshold value) derived from the history of the user's physical condition index to acquire the change over time in them as illustrated in the upper part in FIG. 17. Next, as a comparison, the server 10 calculates the square of the difference between the prediction value and the monitor value as the degree of abnormality, and can obtain the change over time in the abnormal value as illustrated in the lower part in FIG. 17. Further, the server 10 detects the degree of abnormality of the monitor value 700 when the calculated abnormality degree exceeds a preset threshold value (second threshold value). In the present embodiment, the threshold value is set in advance by a medical professional or the like.

In the present embodiment, since the physical condition index (monitor value) is data with high periodicity, it is preferable to use the above-mentioned autoregressive model. However, in the present embodiment, the model used is not limited to the autoregressive model, and may be another model. Further, in the present embodiment, the order of the autoregressive model is not particularly limited, and it is preferable that the order is appropriately optimized. Further, in the present embodiment, the precision of the prediction value may be changed by changing the order of the autoregressive model by the user or the like such as a high-precision estimation mode having a high order and a low-precision mode having a low order.

Returning to FIG. 8 and continuing the explanation, the server 10 then determines whether the monitor value (physical condition index) can be monitored according to the monitoring condition (step S206). When it is determined that the monitor value was monitored according to the monitoring condition (step S206: Yes), the server 10 advances to the processing in step S207. On the other hand, if it is determined that the monitor value is not monitored according to the monitoring condition (step S206: NO), the server 10 advances to the processing in step S209. In detail, the server 10 determines whether the monitor value is monitored after satisfying the monitoring conditions such as the monitor time, the posture of the user when monitoring, the behavior of the user before monitoring (before and after exercise, before and after eating, etc.), the mounting state, and the like based on sensing data from the monitor device 30 (for example, sensing data related to monitor time, user's position information, user's posture, user's behavior, etc.), the user's schedule as the user's attribute information, and the like.

Further, the server 10 determines whether the user has taken the drug (step S207). When it is determined that the user has taken the drug (step S207: Yes), the server 10 advances to the processing in step S208. On the other hand, if it is determined that the user has not taken the drug (step S207: NO), the server 10 advances to the processing in step S209. Specifically, the server 10 may make the above determination based on the user's medication information (presence or absence of medication and medication time) acquired from the medication management system server 60 that manages medication based on the user's medication declaration. At this time, the server 10 may present a medication reminding alert to induce the user to take the drug when it is determined that the user has not taken the medication.

Then, the server 10 presents an abnormality alert to the user (step S208). In the present embodiment, the method of presenting the abnormality alert is not limited, and may be a predetermined image display, a predetermined voice output, a blinking of a light emitting element, a vibration of a vibration module, or the like. Then, after presenting the abnormality alert, the server 10 returns to the processing in step S203.

In the present embodiment, the abnormality alert may be automatically presented not only to the user but also to the user's family. Further, in the present embodiment, when the user is estimated by the server 10 to be in a serious condition based on the monitor value, an abnormality alert may be directly presented to the doctor in charge. In such a case, the alert may be linked with online medical consultation and the like.

Furthermore, the user who is presented with the abnormality alert goes to the family pharmacy alone or with his/her family and presents the history of the physical condition index to the pharmacist. Furthermore, the pharmacist determines whether the cause of the abnormality detection is due to the medication status based on the history of the physical condition index and the medication status (combination of food and drink, combination with over-the-counter drug, etc.). Furthermore, when the pharmacist determines that the cause of the abnormality being detected is a reason other than the medication status, the pharmacist recommends the user to visit a medical institution (at this time, it is preferable that the history of the physical condition index is sent from the family pharmacy to the medical institution via the network 70 of the treatment support system 1.

Returning to FIG. 8 and continuing the explanation, the server 10 does not present the abnormality alert to the user (step S209). Then, the server 10 returns to the processing in step S203.

In the present embodiment, the order of steps S205 to S207 illustrated in FIG. 8 may be changed, and further, the procedure is not limited to performing step S206, and for example, another step may be performed or added instead of it.

Further, in steps S204 and S205, it is determined whether the monitor value exceeds the general-purpose threshold value or the personalization threshold value, but the present embodiment is not limited to this. In the present embodiment, for example, it may be determined whether the monitor value falls below the general-purpose threshold value or the personalization threshold value, or it may be determined whether the monitor value falls within the general-purpose numerical range or the personalized numerical range.

Further, in the treatment support system 1 according to the present embodiment, it is preferable to perform the determination based on the personalization threshold value after the determination based on the general-purpose threshold value. For example, when only the determination based on the personalization threshold value is made, it is not possible to detect the case where the value of the physical condition index is congenitally stable and abnormal (for example, a user whose systolic blood pressure is always stable at 140 mmHg may not be determined as abnormal when compared with the history of the user's physical condition index in the past). Therefore, in the treatment support system 1 according to the present embodiment, it is preferable to perform the determination based on the personalization threshold value after the determination based on the general-purpose threshold value. Further, in a manner as described above, it is possible to determine whether to perform the processing of the next step by the determination based on the general-purpose threshold value, so that the processing load on the treatment support system 1 according to the present embodiment can be reduced.

As described above, according to the above-described present embodiment, the abnormality of the monitor value is detected using the general-purpose threshold value, and the abnormality of the monitor value is evaluated according to the situation of the user, so that it is possible to more appropriately determine whether to visit a medical institution. As a result, according to the present embodiment, it is possible to perform abnormality alerts according to the user's situation, in other words, personalized abnormality alerts. Therefore, while suppressing an increase in unnecessary abnormality alerts, it is possible to avoid an increase in the number of unnecessary visits to the medical institution or the number of emergency outpatient visits or the like. Further, according to the present embodiment, it is possible to avoid an increase in the psychological burden on the user. That is, according to the present embodiment, it is possible to provide the user or the like with the treatment support system 1 having higher effectiveness.

In addition, the treatment support system 1 according to the present embodiment has a configuration in which a family pharmacy or a pharmacist can offer the user's consultation before the user visits a medical institution, so that the burden on the doctor can be reduced and it is possible to enhance the safety and effectiveness of the medication treatment for the user. Furthermore, in the treatment support system 1 according to the present embodiment, since the monitor values are stored in a database, so that it facilitates information sharing between the user, the doctor, and the pharmacist and is helpful in advancing treatment more effectively.

### 2.7 Modifications

The details of the first embodiment of the present disclosure have been described above. Next, respective modifications of the first embodiment will be described.

(First Modification)

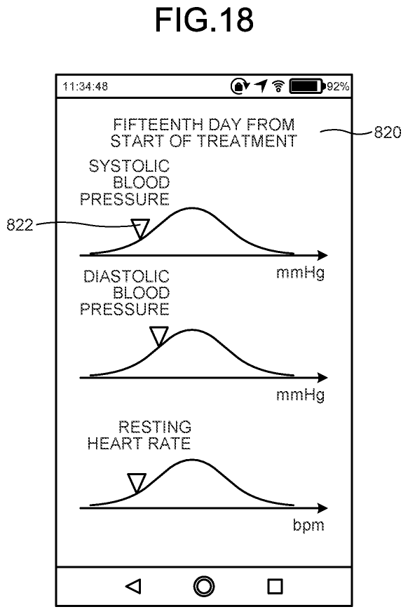

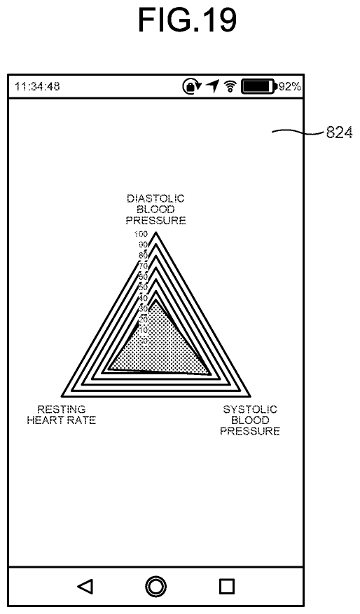

In the first embodiment described above, in order to present the monitor value to the user in an easy-to-understand manner, the monitor value may be presented in a form in which the recovery status of the user can be intuitively understood. Therefore, with reference to FIGS. 18 and 19, a monitor value output example will be described as a modification of the above-described first embodiment. FIGS. 18 and 19 are explanatory views illustrating an example of an output screen 820 and an example of an output screen 824 according to the modification of the present embodiment.

For example, as illustrated in FIG. 18, in the present modification, the monitor value may be presented to the user in the form of a frequency distribution graph. Specifically, a graph of the data distribution of the physical condition index of other users having the attribute information similar to the user's attribute information and at the treatment elapsed date same as that of the user's monitor value is displayed on the output screen 820 illustrated in FIG. 18. Further, an arrow 822 indicating the monitor value is displayed on the output screen 820, so as to be superimposed on the graph of the data distribution. According to such a display, the user can easily compare his/her own monitor value with the situation of other users similar to himself/herself, so that he/she can intuitively grasp his/her recovery situation. In the present modification, the frequency distribution graph is not limited to the normal distribution curve as illustrated in FIG. 18, and may be, for example, a histogram.

Further, for example, as illustrated in FIG. 19, in the present modification, the monitor value may be presented to the user in the form of a radar chart. Specifically, in the present modification, the value of the current user's physical condition index (monitor value) is calculated as a ratio (%) to the treatment target value with the value of the user's physical condition index at the start of treatment is set to the recovery level of 0%, and the treatment target value is set to the recovery level of 100%, and is plotted on the radar chart included in the output screen 824 in FIG. 19. According to such a display, the user can easily compare his/her own monitor value with the situation of other users similar to himself/herself, so that he/she can intuitively grasp his/her recovery situation. Although not illustrated, in the present modification, when a plurality of types of physical condition indexes is monitored, the ratio of the monitor value to the treatment target value is calculated for each type, and the average value thereof is calculated and plotted in chronological order, and may be presented to the user.

(Second Modification)

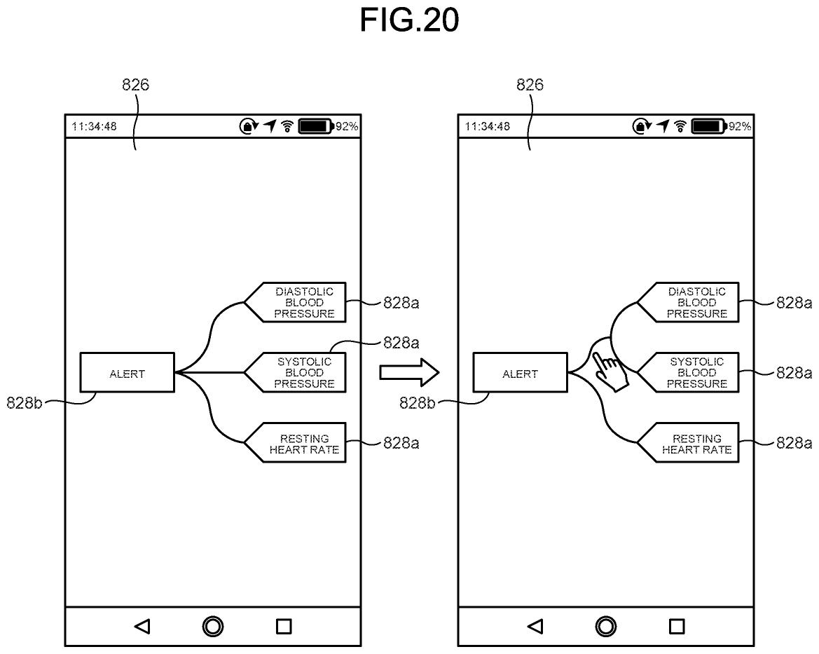

Further, in the first embodiment described above, a plurality of types of physical condition indexes may be set as monitor items. A modification of the conditions for presenting an abnormality alert in such a case will be described with reference to FIG. 20. FIG. 20 is an explanatory diagram illustrating an example of a setting screen 826 according to the modification of the present embodiment.

First, in the present modification, in a case where a plurality of types of physical condition indexes is set as monitor items, when even one of them is detected as abnormal, it is assumed that an abnormality alert is set to be presented to the user (default setting). In the present modification, the medical professional, etc. changes the setting from such default setting, so that an abnormality alert can be presented to the user when an abnormality is detected in a plurality of predetermined types of physical condition indexes instead of one physical condition index.

More specifically, a medical professional or the like can change the setting as described above by performing an input operation on the setting screen 826 as illustrated in FIG. 20. For example, on the left side of FIG. 20, icons 826a of a plurality of types (diastolic blood pressure, systolic blood pressure, heart rate at completion) set as monitor items are displayed, and are each connected to an alert icon 828b by a line. In such a case, when an abnormality is detected in any one of diastolic blood pressure, systolic blood pressure, and resting heart rate, an abnormality alert will be presented. Therefore, as illustrated on the right side of FIG. 20, the medical professional and the like change the line connecting the icons 826a of a plurality of types (diastolic blood pressure, systolic blood pressure, resting heart rate) to the alert icon 828b, so that it is possible to change the condition under which the abnormality alert is presented. Specifically, as illustrated on the right side of FIG. 20, the medical professional and the like connect the diastolic blood pressure icon 828a to the systolic blood pressure icon 828a with a line, and further connect the connected line with the alert icon 828b. By doing so, the abnormality alert will be presented only when an abnormality is detected in both the diastolic blood pressure and the systolic blood pressure. The setting screen 826 illustrated in FIG. 20 is merely an example, and the present modification is not limited to such a screen, and may further include other displays and the like.

Further, in the present modification, the alert level may be set based on the abnormality alert history (for example, stored in the above-mentioned storage unit 200) of other users having attribute information similar to the attribute information on the user. Then, in the present modification, the range in which the abnormality alert is presented according to the alert level may be set step by step such as only the user, only the user and the family of the user, and further, the user, the family of the user, and the medical institution. More specifically, for other users who have attribute information similar to the user's attribute information, the alert level is set higher for the type of physical condition index with more abnormality alerts, and in this case, the abnormality alert is also sent to the medical institution. On the other hand, in the other users mentioned above, the alert level is set lower for the type of physical condition index with fewer abnormality alerts, and in this case, although the abnormality alert is presented to the user, only the monitor value is recorded (stored).

(Third Modification)

Further, in the first embodiment described above, whether the user has taken a drug is s determined by obtaining the user's medication information (whether the drug is taken and the time of medication) from the medication management system server 60 that manages the medication based on the user's medication declaration. However, the present embodiment is not limited to such a method, and other methods may be used. For example, in the present modification, the medication information acquisition unit 138 of the processing unit 110 of the server 10 may include a sensor device that detects a signal from a signal generator built in the internal medicine to be taken by the user. In such a case, the signal generator transmits a predetermined signal by reacting with gastric fluid or the like in the user's body. Then, when the sensor device of the medication information acquisition unit 138 detects the predetermined signal, it is possible to recognize that the user has taken the drug.

Further, the present modification is not limited to the above-mentioned method, and for example, the user causes the sensor device of the medication information acquisition unit 138 read the electronic tag or barcode attached to the prescription drug before taking the drug, so that the server 10 may be made to recognize what kind of drug was taken at what time. Further, in the present modification, when the prescription drug is in a liquid state, the sensor device of the medication information acquisition unit 138 is caused to irradiate the prescription drug with infrared rays and detect the infrared rays transmitted through the prescription drug (for example, the prescription drug can be specified by the wavelength of the infrared rays absorbed by the prescription drug), so that the same can be done as described above.

(Fourth Modification)

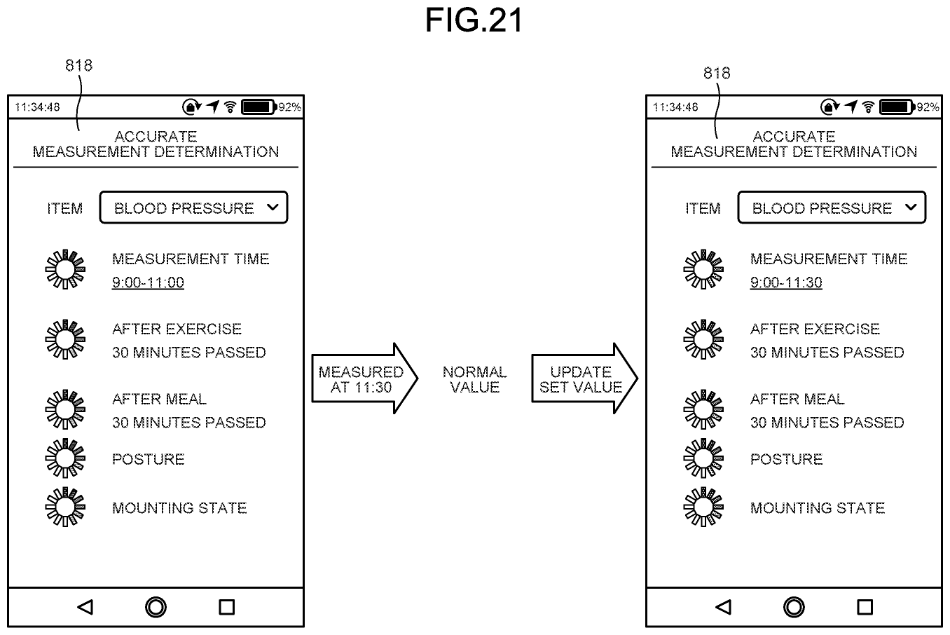

Further, in the first embodiment described above, when the monitor value is not abnormal, that is, when it is a normal value, the preset monitoring condition may be dynamically updated (changed). Therefore, a modification of the present embodiment that dynamically updates the monitoring condition will be described below with reference to FIG. 21. FIG. 21 is an explanatory diagram illustrating an example of the determination screen 818 according to the modification of the present embodiment.

For example, it is assumed that the user's physical condition index is monitored according to part of the monitoring conditions of the determination screen 818 illustrated on the left side of FIG. 21. Then, it is assumed that the monitored user's physical condition index (monitor value) is determined to be a normal value by the method described in the first embodiment. At this time, under the preset monitoring condition, the monitor time was set in the range of 9:00 to 11:00, but the monitor value is actually monitored at 11:30. Therefore, in the present modification, the server 10 learns the monitoring condition in which the normal value is monitored, and the monitor time (set value) included in the monitoring condition is updated dynamically up to the range of 11:30 as illustrated on the determination screen 818 on the right side of FIG. 21.

In the present modification, since the monitoring condition can be dynamically updated in this way, the range of the set value of the monitoring condition can be appropriately widened, and as a result, it is possible to increase the amount of data of the history of the physical condition index that can be appropriately compared with the monitor value. However, in the present modification, in order to give priority to proper monitoring, it is preferable that the set value can be updated only within a range continuous with the preset monitoring condition.

### 3. SECOND EMBODIMENT

In the first embodiment described above, the treatment progress or the treatment back calculation simulation may be performed while the user's physical condition index in step S203 illustrated in FIG. 8 is being monitored. Therefore, as a second embodiment of the present disclosure, an embodiment of such a treatment progress or a treatment back calculation simulation will be described.

In the present embodiment, the configuration of the treatment support system 1 is the same as that of the first embodiment, and the description of the treatment support system 1 and FIG. 2 according to the first present embodiment can be referred to. Therefore, the description of the configuration of the treatment support system 1 according to the present embodiment will be omitted here. Further, in the present embodiment, since the monitor device 30 is also common except for the estimation block 160 of the processing unit 110 of the server 10, the description other than the estimation block 160 will be omitted.

### 3.1 Detailed Configuration of Estimation Block 160

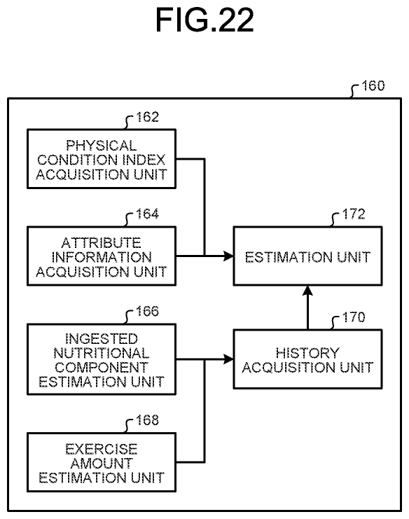

First, with reference to FIG. 22, each functional unit of the estimation block 160 of the processing unit 110 will be sequentially described. FIG. 22 is a diagram illustrating a functional configuration of the estimation block 160 according to the present embodiment. Specifically, as illustrated in FIG. 22, the estimation block 160 of the processing unit 110 includes a physical condition index acquisition unit 162, an attribute information acquisition unit 164, an ingested nutritional component estimation unit (third estimation unit) 166, and an exercise amount estimation unit 168, a history acquisition unit 170, and an estimation unit (first estimation unit, second estimation unit) 172. Each functional unit of the estimation block 160 will be described in sequence below, but since the physical condition index acquisition unit 162, the attribute information acquisition unit 164, and the history acquisition unit 170 are common to the physical condition index acquisition unit 132, the attribute information acquisition unit 134 and the history acquisition unit 146 of the first embodiment, the explanation thereof will be omitted.

(Ingested Nutritional Component Estimation Unit 166)

The ingested nutritional component estimation unit 166 can estimate the nutritional component based on the image of the meal ingested by the user. In detail, the ingested nutritional component estimation unit 166 recognizes the meal contents from the image of the meal ingested by the user by using the learning database obtained by machine learning, and refers to the database based on the recognized meal contents to estimate the nutritional component.

(Exercise Amount Estimation Unit 168)

The exercise amount estimation unit 168 can estimate the amount of exercise of the user based on the sensing data obtained by the motion sensor of the sensor unit 304. Specifically, the exercise amount estimation unit 168 calculates the exercise intensity and exercise time of the user's daily exercise based on the sensing data, and for example, multiplies the calculated exercise intensity by the exercise time to obtain the amount of exercise of the user. Further, the exercise amount estimation unit 168 estimates the activity level of the user by comparing the calculated amount of exercise of the user with the amount of exercise of other users, stored in the database, who have attribute information similar to the user.

(Estimation Unit 172)

The estimation unit 172 can estimate (predict) future changes in the physical condition index of the user, in other words, can perform a treatment progress simulation, based on the history of the physical condition index of other users having attribute information similar to the attribute information on the user. Further, the estimation unit 172 can estimate the management condition imposed on the user in order that the physical condition index (monitor value) of the user reaches the target value, in other words, can perform a treatment back calculation simulation, based on the history of the physical condition index of other users having the attribute information similar to the attribute information on the user.

### 3.2 Information Processing Method

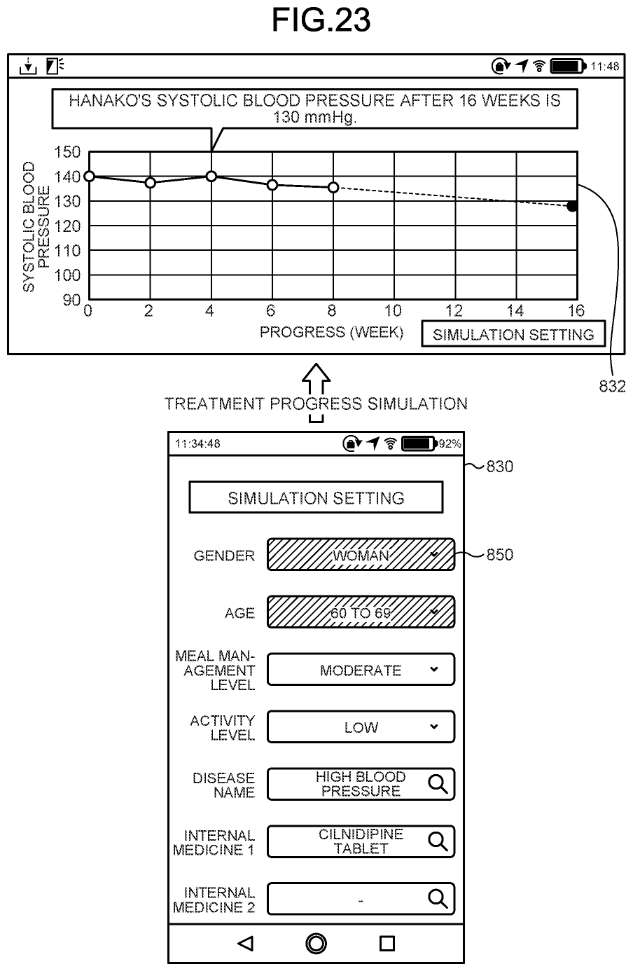

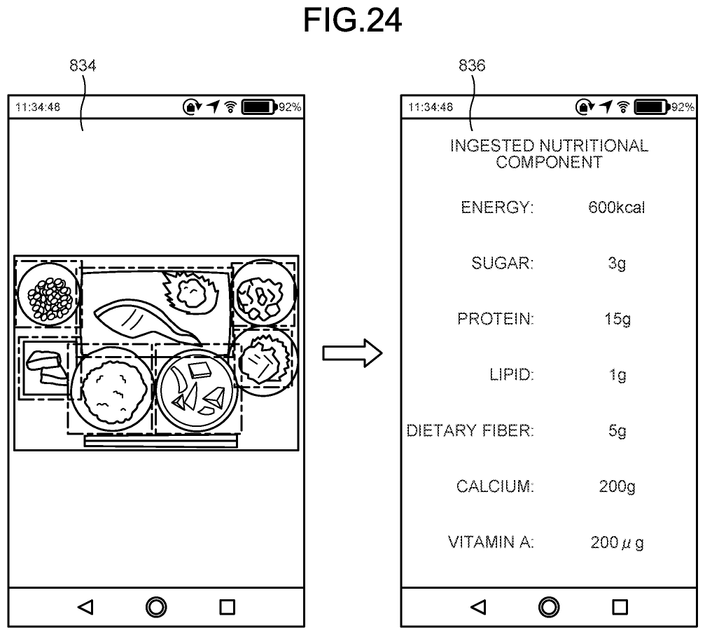

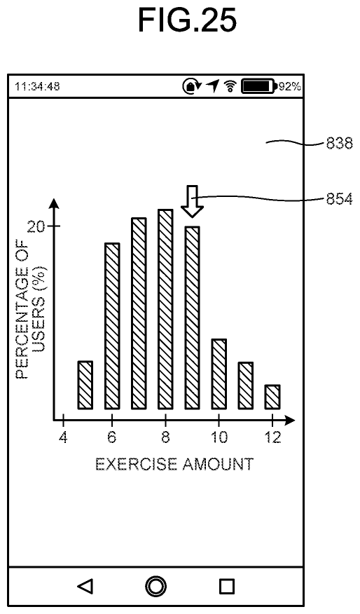

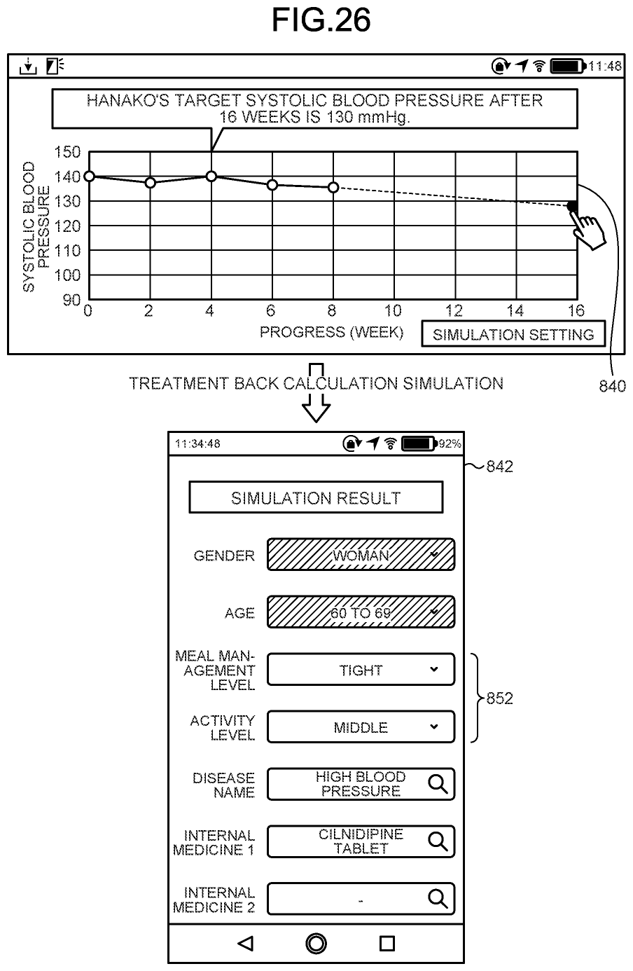

Next, the information processing method according to the second embodiment of the present disclosure will be described with reference to FIGS. 23 to 26. FIGS. 23 and 26 are explanatory views for explaining the estimation method according to the present embodiment. Further, FIG. 24 is an explanatory diagram for explaining a method of estimating the ingested nutritional component according to the present embodiment, and FIG. 25 is an explanatory diagram illustrating an example of an activity level display screen 838 according to the present embodiment. The treatment progress simulation and the treatment back calculation simulation according to the present embodiment can be appropriately performed by the user or the like during the monitoring in step S203 illustrated in FIG. 8.

—Treatment Progress Simulation—

First, the treatment progress simulation according to the present embodiment will be described with reference to FIGS. 23 to 25. First, the user or the like inputs attribute information, information on the drug being taken, meal management level, activity level, and the like for setting items 850 of a simulation setting screen 830 as illustrated in the lower part in FIG. 23 (in the present embodiment, these may be automatically set, and the details of the automatic setting such as the meal management level and the activity level will be described later). The input information can be used when extracting the history of the physical condition index of other users used in performing the treatment progress simulation. Further, the input information also serves as a precondition for the treatment progress simulation, and when the user or the like inputs the information on the drug being taken, the treatment progress simulation when the drug is taken is performed. On the other hand, when the user or the like does not input the information on the drug being taken, the treatment progress simulation when the drug is not taken is performed.

First, the server 10 extracts the history of the physical condition index of other users having attribute information and the like similar to the attribute information, medication information, meal management level, activity level, and the like of the user, based on the input information to machine learn the history of the physical condition index of other users extracted as training data, and generates an estimation model. Further, the server 10 calculates the value of the physical condition index at the subsequent date (treatment elapsed date) input in advance from the user from the generated estimation model. The server 10 presents the calculated value to the user, for example, as a simulation result display screen 832 as illustrated in the upper part in FIG. 23.

Further, the automatic setting of the meal management level in the present embodiment will be described. The server 10 recognizes the meal contents by using the learning database obtained by machine learning from a meal image 834 of the meal ingested by the user illustrated on the left side of FIG. 24, and refers to the database based on the recognized meal contents to estimate the nutritional component. The server 10 can present the estimated nutritional component to the user on an ingested nutritional component result screen 836 as illustrated on the right side of FIG. 24, for example. Further, the server 10 compares, for example, the estimated ingested nutritional component with the meal ingestion standard or the like of the Ministry of Health, Labor and Welfare, and based on the ratio of the estimated nutritional component to the recommended amount recommended by the meal ingestion standard, calculates the meal management level. It should be noted that the present embodiment is not limited to the estimation of the ingested nutritional component by image recognition. For example, in the present embodiment, when an electronic tag is attached to the container like a meal provided in a cafeteria in a school or a company, and the electronic tag stores information on meal contents and nutritional components, the server 10 may estimate the ingested nutritional component by taking in the information in the electronic tag.

Next, the automatic setting of the activity level in the present embodiment will be described. The server 10 calculates the exercise intensity and exercise time of the user's daily exercise based on the sensing data by the motion sensor of the sensor unit 304, and for example, multiplies the calculated exercise intensity by the exercise time to calculate the exercise amount of the user. Further, the exercise amount estimation unit 168 estimates the activity level of the user by comparing the calculated amount of exercise of the user with the amount of exercise of other users, stored in the database, who have attribute information similar to the user. For example, the server 10 indicates the estimated amount of exercise of the user with an arrow 854 on the histogram illustrating the distribution of the amount of exercise of other users of the activity level display screen 838 as illustrated in FIG. 25. Further, the server 10 compares the estimated amount of exercise of the user with the distribution of the amount of exercise of other users, and estimates the activity level.

—Treatment Back Calculation Simulation—

Next, the treatment back calculation simulation according to the present embodiment will be described with reference to FIG. 26. First, the user inputs the value of the physical condition index that he/she targets on the simulation setting screen 840 as illustrated in the lower part in FIG. 26 (specifically, by moving the cursor, a target value and the treatment elapsed date at which the user wants to reach the target value can be input).

Then, the server 10 extracts the history of the physical condition index, of other users, which is similar to the user's attribute information (gender, age), where the other users are patients with the disease same as the disease of the user, and machine learns the history of the physical condition index of the other users as training data, to generate an estimation model. Further, the server 10 estimates the management condition (for example, the meal management level and the activity level) for the user to reach the target value from the generated estimation model. The server 10 presents the estimated management condition to the user, for example, as a simulation result display screen 842 as illustrated in the lower part in FIG. 26. On the simulation result display screen 842, the meal management level, activity level, and the like that the user must perform in order to reach the target value input by the user are illustrated as management items 852.

In the present embodiment, when the setting of the target value is changed, the treatment back calculation simulation is automatically started, and the management condition is re-estimated again. Further, in the present embodiment, it is preferable that the target value can be set only within the range that can be reached by changing the meal management level and the activity level.

As described above, according to the above-described present embodiment, the treatment progress simulation and the treatment back calculation simulation provide useful information for the user to maintain motivation for home treatment and useful information for effectively advancing home treatment. That is, according to the present embodiment, it is possible to provide the user or the like with the treatment support system 1 having higher effectiveness.

### 4. EXAMPLE

The details of the first and second embodiments of the present disclosure have been described above. Next, the example of the information processing method of the present embodiment will be described more specifically while illustrating a specific example. The examples illustrated below are merely an example of the information processing methods according to the first and second embodiments, and the information processing methods according to the first and second embodiments are not limited to the following examples.

### 4.1 First Example

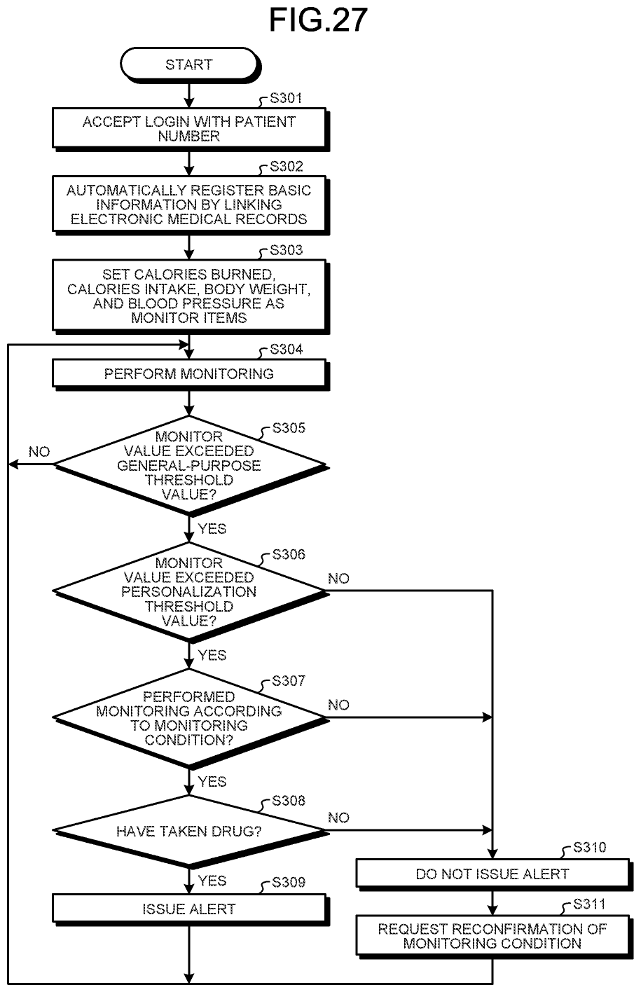

First, the first example, which is a use case when the user is a hypertensive patient, will be described with reference to FIG. 27. FIG. 27 is a flowchart of the first example of the present disclosure. As illustrated in FIG. 27, in the embodiment, the steps from step S301 to step S311 can be mainly included. The details of each of these steps according to the embodiment will be described below. In the embodiment described below, steps S304 to S311 are repeatedly performed until the user is completely cured.

First, the user visits a medical institution, receives guidance on modifying lifestyle habits from a doctor, and sets a treatment target. At this time, in the embodiment, it is assumed that the user has not been prescribed an antihypertensive drug.

Next, the user starts home treatment while using the treatment support system 1 of the present embodiment. First, the server 10 accepts login by the user's patient number (step S301). Then, the server 10 automatically registers the basic information on the user based on the patient number in cooperation with the electronic medical records (step S302). Then, the server 10 sets the calories burned, calorie intake, body weight, and blood pressure as monitor items (step S303). Then, the server 10 monitors the monitor items set in step S303 (step S304).

Then, the server 10 determines whether the monitor value exceeds the general-purpose threshold value (step S305). When it is determined that the monitor value exceeds the general-purpose threshold value (step S305: Yes), the server 10 advances to the processing in step S306. On the other hand, when it is determined that the monitor value does not exceed the general-purpose threshold value (step S305: NO), the server 10 returns to the processing in step S304 and continues monitoring. Further, the server 10 determines whether the monitor value exceeds the personalization threshold value (step S306). When it is determined that the monitor value exceeds the personalization threshold value (step S306: Yes), the server 10 advances to the processing in step S307. On the other hand, if it is determined that the monitor value does not exceed the personalization threshold value (step S306: NO), the server 10 advances to the processing in step S310.

Next, the server 10 determines whether the monitor value can be monitored according to the monitoring condition (step S307). When it is determined that the monitor value was monitored according to the monitoring condition (step S307: Yes), the server 10 advances to the processing in step S308. On the other hand, if it is determined that the monitor value is not monitored according to the monitoring condition (step S307: NO), the server 10 advances to the processing in step S310.

Further, the server 10 determines whether the user has taken the drug (step S308). When it is determined that the user has taken the drug (step S308: Yes), the server 10 advances to the processing in step S309. On the other hand, if it is determined that the user has not taken the drug (step S308: NO), the server 10 advances to the processing in step S310.

Then, the server 10 presents an abnormality alert to the user (step S309). Then, after presenting the abnormality alert, the server 10 returns to the processing in step S304.

On the other hand, the server 10 does not present the abnormality alert to the user (step S310). Then, the server 10 advances to the processing in step S311. Further, the server 10 performs a display requesting reconfirmation of the monitoring conditions, such as presenting the determination screen 818 to the user (step S311).

In the embodiment, the blood pressure, which is the monitor value, exceeded the general-purpose threshold value but did not exceed the personalization threshold value during the first one month from the start of the home treatment, so that by the processing in step S305 and step S306 described above, no abnormality alert was presented.

Therefore, the user has reviewed his eating habits and exercise habits and his blood pressure has been steadily decreasing, but one day, just before the monitor, the user has eaten and smoked contrary to the monitoring condition. In the embodiment, the server 10 can automatically recognize that the user has eaten or smoked by image recognition or a motion sensor. Then, when the server 10 performed monitoring in the processing in step S304 described above, the blood pressure, which is the monitor value, exceeded both the general-purpose threshold value and the personalization threshold value (steps S305 and S306). Further, in step S307, since the user has eaten/smoked immediately before the monitor, the server 10 determines that the user is not monitored according to the monitoring conditions, and although an abnormality alert is not issued (step S310), for example, presents the determination screen 818 to the user, and performs a display requesting reconfirmation of the monitoring conditions in order to notify that the user is not monitored according to the monitoring conditions (step S311).

Furthermore, the user continued home treatment as before from the next day onward, and the monitor value reached the treatment target.

As described above, in the present embodiment, when the monitor value exceeds the general-purpose threshold value and the personalization threshold value due to the user's behavior (eating/smoking), the server 10 does not detect the result as an abnormality because it is clear that it is not the result due to an abnormality in the user's body. Therefore, according to the embodiment, it is possible to appropriately determine whether to visit a medical institution by evaluating the monitor value according to the situation of the user. As a result, according to the present embodiment, it is possible to perform abnormality alerts according to the user's situation, in other words, personalized abnormality alerts. Therefore, while suppressing an increase in unnecessary abnormality alerts, it is possible to avoid an increase in the number of unnecessary visits to the medical institution or the number of emergency outpatient visits or the like.

### 4.2 Second Example

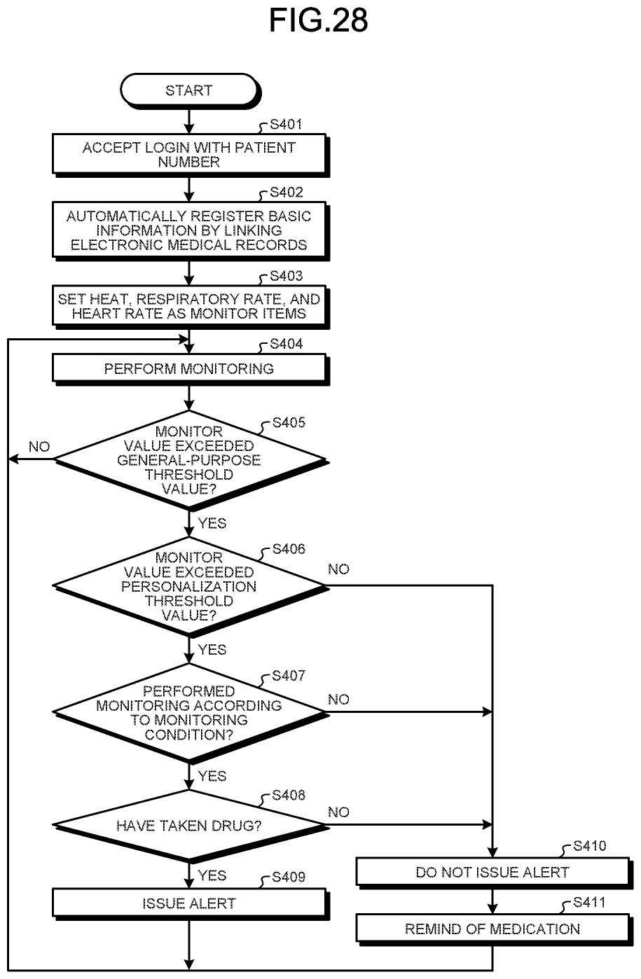

Next, with reference to FIG. 28, the second example, which is a use case when the user is a patient with bacterial pneumonia, will be described. FIG. 28 is a flowchart of the second example of the present disclosure. As illustrated in FIG. 28, in the embodiment, the steps from step S401 to step S411 can be mainly included. The details of each of these steps according to the embodiment will be described below. In the embodiment described below, steps S404 to S411 are repeatedly performed until the user is completely cured.

First, in the present embodiment, the user visits a medical institution, receives a prescription of an antibacterial drug from a doctor, and starts home treatment while using the treatment support system 1 of the present embodiment.

In addition, since steps S401 to S410 in FIG. 28 of the embodiment are similar to steps S301 to S310 in FIG. 27 except that the server 10 sets heat, respiratory rate, and heart rate as monitor items in step S403, detailed description of these steps will be omitted here. In addition, the server 10 presents the user with a medication remind notification that induces medication (step S411).

In the embodiment, the monitor value exceeded the general-purpose threshold value but did not exceed the personalization threshold value for the first few days from the start of the home treatment, so that by the processing in step S405 and step S406 described above, no abnormality alert was presented. Therefore, the user stopped taking the antibacterial drug at his/her own discretion. A few days after discontinuing the drug, the bacteria infecting the user acquired drug resistance and the symptoms of the user's bacterial pneumonia recurred.

Then, when the server 10 performed monitoring in the processing in step S304 described above, the monitor value exceeds both the general-purpose threshold value and the personalization threshold value (steps S405 and S406). Then, in step S408, the server 10 does not issue an abnormality alert based on the fact that the user has not taken the antibacterial drug (step S410), but presents a notification of medication reminding to the user (step S411). By doing so, in the embodiment, the user is guided to take appropriate medication.

Then, the user started taking the antibacterial drug again based on the medication reminding, but the infected bacteria has acquired drug resistance, and the prescribed antibacterial drug was ineffective, so that the symptoms of bacterial pneumonia have continued. As a result, when monitoring was performed in the processing in step S304 described above, the monitor value exceeded both the general-purpose threshold value and the personalization threshold value (steps S405 and S406). After that, the server 10 issued an abnormality alert (step S409), so that the user visited a medical institution.

As described above, in the present embodiment, when the monitor value exceeds the general-purpose threshold value and the personalization threshold value due to the user's behavior (stopping medication), the server 10 does not detect the result as an abnormality because it is clear that it is not the result due to an abnormality in the user's body. Therefore, according to the embodiment, it is possible to appropriately determine whether to visit a medical institution by evaluating the monitor value according to the situation of the user. As a result, according to the present embodiment, it is possible to perform abnormality alerts according to the user's situation, in other words, personalized abnormality alerts. Therefore, while suppressing an increase in unnecessary abnormality alerts, it is possible to avoid an increase in the number of unnecessary visits to the medical institution or the number of emergency outpatient visits or the like.

### 5. SUMMARY

As described above, according to the above-described embodiments of the present disclosure, not only the abnormality of the monitor value (physical condition index) is detected by using the general-purpose threshold value, but also the monitor value is evaluated according to the user's situation, so that it is possible to appropriately determine whether to visit a medical institution. As a result, according to the present embodiment, it is possible to perform abnormality alerts according to the user's situation, in other words, personalized abnormality alerts. Therefore, while suppressing an increase in unnecessary abnormality alerts, it is possible to avoid an increase in the number of unnecessary visits to the medical institution or the number of emergency outpatient visits or the like. Further, according to the present embodiment, it is possible to avoid an increase in the psychological burden on the user. That is, according to the present embodiment, it is possible to provide the user or the like with the treatment support system 1 having higher effectiveness.

In addition, the treatment support system 1 according to the present embodiment has a configuration in which a family pharmacy or a pharmacist can offer the user's consultation before the user visits a medical institution, so that the burden on the doctor can be reduced and it is possible to enhance the safety and effectiveness of the medication treatment for the user. Furthermore, in the treatment support system 1 according to the present embodiment, since the monitor values of the user are stored in a database, so that it facilitates information sharing between the user, the doctor, and the pharmacist and is helpful in advancing treatment more effectively.

### 6. HARDWARE CONFIGURATION

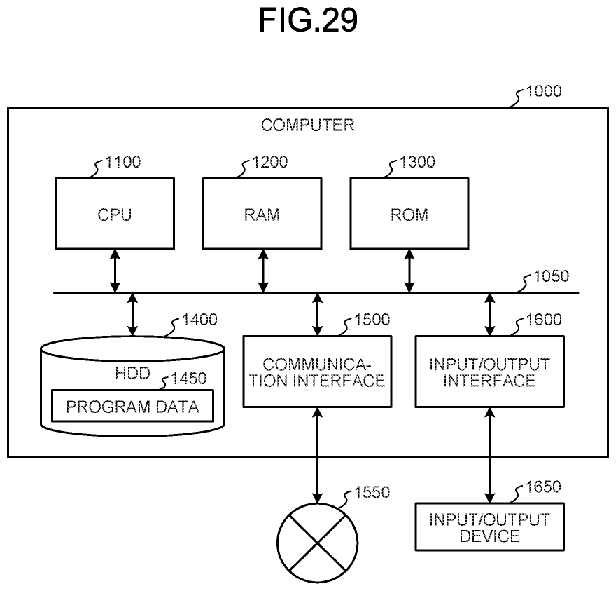

The information processing device such as a server 10 and the like according to each embodiment described above is implemented by, for example, a computer 1000 having a configuration as illustrated in FIG. 29. Hereinafter, the server 10 of the embodiment of the present disclosure will be described as an example. FIG. 29 is a hardware configuration diagram illustrating an example of the computer 1000 that implements the functions of the server 10. The computer 1000 includes a CPU 1100, a RAM 1200, a read only memory (ROM) 1300, a hard disk drive (HDD) 1400, a communication interface 1500, and an input/output interface 1600. Respective units of the computer 1000 are connected by a bus 1050.

The CPU 1100 operates based on a program stored in the ROM 1300 or the HDD 1400, and controls each unit. For example, the CPU 1100 develops a program stored in the ROM 1300 or the HDD 1400 in the RAM 1200, and executes processing corresponding to various programs.

The ROM 1300 stores a boot program such as a basic input output system (BIOS) executed by the CPU 1100 when the computer 1000 is activated, a program depending on hardware of the computer 1000, and the like.

The HDD 1400 is a computer-readable recording medium that non-transiently records programs executed by the CPU 1100, data used by the programs, and the like. Specifically, the HDD 1400 is a recording medium that records an image processing program according to the present disclosure which is an example of program data 1450.

The communication interface 1500 is an interface for the computer 1000 to be connected to an external network 1550 (for example, the Internet). For example, the CPU 1100 receives data from another device or transmits data generated by the CPU 1100 to another device via the communication interface 1500.

The input/output interface 1600 is an interface for connecting an input/output device 1650 to the computer 1000. For example, the CPU 1100 receives data from an input device such as a keyboard and a mouse via the input/output interface 1600. In addition, the CPU 1100 transmits data to an output device such as a display, a speaker, or a printer via the input/output interface 1600. Furthermore, the input/output interface 1600 may function as a media interface that reads a program or the like recorded in a predetermined recording medium (medium). The medium is, for example, an optical recording medium such as a digital versatile disc (DVD) or a phase change rewritable disk (PD), a magneto-optical recording medium such as a magneto-optical disk (MO), a tape medium, a magnetic recording medium, a semiconductor memory, or the like.

For example, in a case where the computer 1000 functions as the server 10 according to the embodiment of the present disclosure, the CPU 1100 of the computer 1000 executes the program stored in the RAM 1200 to implement the functions of the processing unit 110 and the like. Further, the HDD 1400 stores an image processing program or the like according to the present disclosure. The CPU 1100 reads the program data 1450 from the HDD 1400 and executes the program data, but as another example, the program may be acquired from another device via the external network 1550.

Further, the information processing device according to the present embodiment may be applied to a system including a plurality of devices, which is premised on connection to a network (or communication between each device), such as cloud computing. That is, the information processing device according to the present embodiment described above can be realized as, for example, an information processing system that performs processing according to the image processing method according to the present embodiment by a plurality of devices.

### 7. SUPPLEMENT

The present embodiment of the present disclosure described above may include, for example, a program for making a computer function as an information processing device according to the present embodiment, and a non-temporary tangible medium in which the program is recorded. Further, the program may be distributed via a communication line (including radio communication) such as the Internet.

In addition, each step in the image processing in each of the above-described embodiments does not necessarily have to be processed in the order described. For example, each step may be processed in an appropriately reordered manner. Further, each step may be partially processed in parallel or individually instead of being processed in chronological order. Further, the processing method of each step does not necessarily have to be processed according to the described method, and may be processed by another method by another functional unit, for example.

The preferred embodiments of the present disclosure have been described in detail with reference to the accompanying drawings, but the technical scope of the present disclosure is not limited to such examples. It is obvious that those skilled in the art in the technical field of the present disclosure can find various revisions and modifications within the scope of a technical concept described in claims, and it should be understood that these revisions and modifications will also be naturally come under the technical scope of the present disclosure.

Furthermore, the effects described in the present specification are merely illustrative or exemplified effects, and are not limitative. That is, the technique according to the present disclosure can accomplish other effects apparent to those skilled in the art from the description of the present specification, in addition to or instead of the effects described above.

The present technology may also be configured as below.

(1) An information processing device comprising:

a physical condition index acquisition unit that acquires, from a monitor device that monitors one or more physical condition indexes of a user, the physical condition indexes;

an attribute information acquisition unit that acquires attribute information on the user;

a medication information acquisition unit that acquires a medication information on the user;

a comparison unit that compares the physical condition indexes with a preset first threshold value;

an evaluation unit that refers to a history of the physical condition indexes of the user or another user selected based on at least one of the attribute information and the medication information according to a result of the comparison to evaluate the physical condition indexes; and

an output unit that outputs predetermined information according to a result of the evaluation.

(2) The information processing device according to (1), further comprising:

a storage unit that stores a history of the physical condition indexes of a plurality of other users; and

a history acquisition unit that acquires the history of the physical condition indexes of the other users having attribute information similar to the attribute information on the user from the storage unit, wherein

the evaluation unit evaluates the physical condition indexes by comparing the physical condition indexes with the history of the physical condition indexes of the other users.

(3) The information processing device according to (2), wherein

when the physical condition indexes deviates from a distribution of the physical condition indexes of the other users, the evaluation unit evaluates the physical condition indexes as abnormal.

(4) The information processing device according to (2) or (3), further comprising:

a first estimation unit that estimates the future physical condition indexes of the user based on a history of the physical condition indexes of the other users having attribute information similar to attribute information on the user.

(5) The information processing device according to any one of (2) to (4), further comprising:

a second estimation unit that estimates a management condition imposed on the user for the physical condition indexes of the user to reach a target value based on the history of the physical condition indexes of the other users having attribute information similar to attribute information on the user.

(6) The information processing device according to (4), further comprising:

a third estimation unit that estimates a nutritional component based on an image of a meal ingested by the user, wherein

the first estimation unit estimates the future physical condition indexes of the user based on the estimated nutritional component.

(7) The information processing device according to (1), further comprising:

a storage unit that stores the history of the physical condition indexes of the user; and

a history acquisition unit that acquires the history of the physical condition indexes of the user from the storage unit, wherein

the evaluation unit performs evaluation by comparing the physical condition indexes with the history of the physical condition indexes of the user.

(8) The information processing device according to (7), further comprising:

a model generation unit that estimates an autoregressive model from the history of the physical condition indexes of the user, wherein

the evaluation unit performs evaluation by comparing the physical condition indexes with a prediction value calculated based on the autoregressive model.

(9) The information processing device according to (8), wherein

the evaluation unit

calculates a difference between the physical condition indexes and the prediction value, and

evaluates the physical condition indexes as abnormal when the difference exceeds a preset second threshold value.

(10) The information processing device according to (3), wherein

when the physical condition index acquisition unit acquires a plurality of types of the physical condition indexes,

the output unit outputs the predetermined information according to a type of the physical condition index that is evaluated as abnormal by the evaluation unit.

(11) The information processing device according to (10), wherein

the predetermined information includes an image of a frequency distribution graph or a radar chart.

(12) The information processing device according to (1), further comprising:

a medical record information acquisition unit that acquires medical record information from a medical record management device that manages a medical record created by a medical professional; and

a type determination unit that determines a type of the physical condition index acquired by the physical condition index acquisition unit based on the medical record information.

(13) The information processing device according to any one of (1) to (12), further comprising:

a determination unit that determines whether the physical condition indexes are monitored under a predetermined measurement condition, wherein

the evaluation unit refers to a result of a determination by the determination unit to evaluate the monitored physical condition indexes.

(14) The information processing device according to (13), wherein

the determination unit makes a determination based on a time when the physical condition indexes are monitored, a mounting state of the monitor device, or a posture or an activity state of the user.

(15) The information processing device according to (13) or (14), further comprising:

a condition changing unit that dynamically changes the predetermined measurement condition according to an evaluation of the physical condition indexes.

(16) The information processing device according to any one of (1) to (15), wherein

the medication information acquisition unit acquires the medication information from a medication management device that manages medication based on a medication declaration by the user.

(17) The information processing device according to any one of (1) to (15), wherein

the medication information acquisition unit includes a sensor device that detects a signal from a signal generator built in an internal medicine.

(18) An information processing system comprising:

a monitor device that monitors one or more physical condition indexes on a user; and

an information processing device, wherein

the information processing device includes

a physical condition index acquisition unit that acquires, from the monitor device, the physical condition indexes,

an attribute information acquisition unit that acquires attribute information on the user,

a medication information acquisition unit that acquires medication information on the user,

a comparison unit that compares the physical condition indexes with a preset first threshold value,

an evaluation unit that refers to a history of the physical condition indexes on the user or another user selected based on at least one of the attribute information and the medication information according to a result of the comparison to evaluate the physical condition indexes, and

an output unit that outputs predetermined information according to a result of the evaluation.

(19) An information processing method comprising:

acquiring, from a monitor device that monitors one or more physical condition indexes on a user, the physical condition indexes;

acquiring attribute information on the user;

acquiring a medication information on the user;

comparing the physical condition indexes with a preset first threshold value;

referring to a history of the physical condition indexes on the user or another user selected based on at least one of the attribute information and the medication information according to a result of the comparison to evaluate the physical condition indexes; and

outputting predetermined information according to a result of the evaluation.

### REFERENCE SIGNS LIST

1 TREATMENT SUPPORT SYSTEM
10 SERVER
30, 30a MONITOR DEVICE
32 BAND PORTION
34 CONTROL UNIT
40 USER TERMINAL
50 ELECTRONIC MEDICAL RECORD SYSTEM SERVER
60 MEDICATION MANAGEMENT SYSTEM SERVER
70 NETWORK
100, 300 INPUT UNIT
110 PROCESSING UNIT
120 MONITOR DETERMINATION BLOCK
122 MEDICAL RECORD INFORMATION ACQUISITION UNIT
124 TYPE DETERMINATION UNIT
126 DEVICE CONTROLLER
130 EVALUATION BLOCK
132, 162 PHYSICAL CONDITION INDEX ACQUISITION UNIT
134, 164 ATTRIBUTE INFORMATION ACQUISITION UNIT
136 MONITOR STATE INFORMATION ACQUISITION UNIT
138 MEDICATION INFORMATION ACQUISITION UNIT
140 COMPARISON UNIT
142 DETERMINATION UNIT
144 EVALUATION UNIT
146, 170 HISTORY ACQUISITION UNIT
148 MODEL GENERATION UNIT
150 CONDITION CHANGING UNIT
160 ESTIMATION BLOCK
166 INGESTED NUTRITIONAL COMPONENT ESTIMATION UNIT
168 EXERCISE AMOUNT ESTIMATION UNIT
172 ESTIMATION UNIT
180, 308 COMMUNICATION UNIT
190, 310 OUTPUT UNIT
200, 306 STORAGE UNIT
302 CONTROLLER
304 SENSOR UNIT
700 MONITOR VALUE
702 DISTRIBUTION
800 LOGIN SCREEN
802, 804 BUTTON
806 INPUT SCREEN
808 INPUT FIELD
810 MANAGEMENT SCREEN
812 MONITOR ITEM SETTING SCREEN
814 MONITOR ITEM
816 MONITOR DEVICE MANAGEMENT SCREEN
818 DETERMINATION SCREEN
820, 824 OUTPUT SCREEN
822, 854 ARROW
826 SETTING SCREEN
828a, 828b ICON
830, 840 SIMULATION SETTING SCREEN
832, 842 SIMULATION RESULT DISPLAY SCREEN
834 MEAL IMAGE
836 INGESTED NUTRITIONAL COMPONENT RESULT SCREEN
838 ACTIVITY LEVEL DISPLAY SCREEN
850 SETTING ITEM
852 MANAGEMENT ITEM

1. An information processing device comprising:
a physical condition index acquisition unit that acquires, from a monitor device that monitors one or more physical condition indexes of a user, the physical condition indexes;
an attribute information acquisition unit that acquires attribute information on the user;
a medication information acquisition unit that acquires a medication information on the user;
a comparison unit that compares the physical condition indexes with a preset first threshold value;
an evaluation unit that refers to a history of the physical condition indexes of the user or another user selected based on at least one of the attribute information and the medication information according to a result of the comparison to evaluate the physical condition indexes; and
an output unit that outputs predetermined information according to a result of the evaluation.

2. The information processing device according to claim 1, further comprising:
a storage unit that stores a history of the physical condition indexes of a plurality of other users; and
a history acquisition unit that acquires the history of the physical condition indexes of the other users having attribute information similar to the attribute information on the user from the storage unit, wherein
the evaluation unit evaluates the physical condition indexes by comparing the physical condition indexes with the history of the physical condition indexes of the other users.

3. The information processing device according to claim 2, wherein
when the physical condition indexes deviates from a distribution of the physical condition indexes of the other users, the evaluation unit evaluates the physical condition indexes as abnormal.

4. The information processing device according to claim 2, further comprising:
a first estimation unit that estimates the future physical condition indexes of the user based on a history of the physical condition indexes of the other users having attribute information similar to attribute information on the user.

5. The information processing device according to claim 2, further comprising:
a second estimation unit that estimates a management condition imposed on the user for the physical condition indexes of the user to reach a target value based on the history of the physical condition indexes of the other users having attribute information similar to attribute information on the user.

6. The information processing device according to claim 4, further comprising:
a third estimation unit that estimates a nutritional component based on an image of a meal ingested by the user, wherein
the first estimation unit estimates the future physical condition indexes of the user based on the estimated nutritional component.

7. The information processing device according to claim 1, further comprising:
a storage unit that stores the history of the physical condition indexes of the user; and
a history acquisition unit that acquires the history of the physical condition indexes of the user from the storage unit, wherein
the evaluation unit performs evaluation by comparing the physical condition indexes with the history of the physical condition indexes of the user.

8. The information processing device according to claim 7, further comprising:
a model generation unit that estimates an autoregressive model from the history of the physical condition indexes of the user, wherein
the evaluation unit performs evaluation by comparing the physical condition indexes with a prediction value calculated based on the autoregressive model.

9. The information processing device according to claim 8, wherein
the evaluation unit
calculates a difference between the physical condition indexes and the prediction value, and
evaluates the physical condition indexes as abnormal when the difference exceeds a preset second threshold value.

10. The information processing device according to claim 3, wherein
when the physical condition index acquisition unit acquires a plurality of types of the physical condition indexes,
the output unit outputs the predetermined information according to a type of the physical condition index that is evaluated as abnormal by the evaluation unit.

11. The information processing device according to claim 10, wherein
the predetermined information includes an image of a frequency distribution graph or a radar chart.

12. The information processing device according to claim 1, further comprising:
a medical record information acquisition unit that acquires medical record information from a medical record management device that manages a medical record created by a medical professional; and
a type determination unit that determines a type of the physical condition index acquired by the physical condition index acquisition unit based on the medical record information.

13. The information processing device according to claim 1, further comprising:
a determination unit that determines whether the physical condition indexes are monitored under a predetermined measurement condition, wherein
the evaluation unit refers to a result of a determination by the determination unit to evaluate the monitored physical condition indexes.

14. The information processing device according to claim 13, wherein
the determination unit makes a determination based on a time when the physical condition indexes are monitored, a mounting state of the monitor device, or a posture or an activity state of the user.

15. The information processing device according to claim 13, further comprising:
a condition changing unit that dynamically changes the predetermined measurement condition according to an evaluation of the physical condition indexes.

16. The information processing device according to claim 1, wherein
the medication information acquisition unit acquires the medication information from a medication management device that manages medication based on a medication declaration by the user.

17. The information processing device according to claim 1, wherein
the medication information acquisition unit includes a sensor device that detects a signal from a signal generator built in an internal medicine.

18. An information processing system comprising:
a monitor device that monitors one or more physical condition indexes on a user; and
an information processing device, wherein
the information processing device includes
a physical condition index acquisition unit that acquires, from the monitor device, the physical condition indexes,
an attribute information acquisition unit that acquires attribute information on the user,
a medication information acquisition unit that acquires medication information on the user,
a comparison unit that compares the physical condition indexes with a preset first threshold value,
an evaluation unit that refers to a history of the physical condition indexes on the user or another user selected based on at least one of the attribute information and the medication information according to a result of the comparison to evaluate the physical condition indexes, and
an output unit that outputs predetermined information according to a result of the evaluation.

19. An information processing method comprising:
acquiring, from a monitor device that monitors one or more physical condition indexes on a user, the physical condition indexes;
acquiring attribute information on the user;
acquiring a medication information on the user;
comparing the physical condition indexes with a preset first threshold value;
referring to a history of the physical condition indexes on the user or another user selected based on at least one of the attribute information and the medication information according to a result of the comparison to evaluate the physical condition indexes; and
outputting predetermined information according to a result of the evaluation.
